(19) United States
(12) Patent Application Publication
(10) Pub. No.: US 2016/0198966 A1
(43) Pub. Date: Jul. 14, 2016

(54) BIOLOGICAL INFORMATION MEASURING MODULE, BIOLOGICAL INFORMATION MEASURING APPARATUS, LIGHT DETECTING APPARATUS, LIGHT DETECTING MODULE, AND ELECTRONIC APPARATUS

(71) Applicant: Seiko Epson Corporation, Tokyo (JP)

(72) Inventors: Akira Uematsu, Suwa-shi (JP); Akira Inagaki, Matsumoto-shi (JP); Atsushi Matsuo, Tachikawa-shi (JP); Hironori Hasei, Azumino-shi (JP)

(21) Appl. No.: 14/984,991

(22) Filed: Dec. 30, 2015

(30) Foreign Application Priority Data
Jan. 13, 2015 (JP) 2015-003905
Mar. 9, 2015 (JP) 2015-045582

(51) Int. Cl. A61B 5/024; H01L 31/0216; H01L 31/02; A61B 5/026

(52) U.S. Cl. CPC A61B 5/02444; A61B 5/02438; A61B 5/0261; H01L 31/02162; H01L 31/02005

(57) ABSTRACT

A biological information measuring module includes a light emitting unit that emits light, a light receiving unit that receives the light via an object, and a multilayered film optical filter that is provided on the light receiving unit on a side where the light is incident. The multilayered film optical filter is constituted by a laminated body of five or more layers and 120 or less layers.

### CROSS-REFERENCES TO RELATED APPLICATIONS

This application claims priority to Japanese Patent Applications No. 2015-003905, filed Jan. 13, 2015, and No. 2015-045582, filed Mar. 9, 2015, all of which are hereby incorporated by reference.

### BACKGROUND

1. Technical Field

The present invention relates to a biological information measuring module, and a biological information measuring apparatus, a light detecting apparatus, a light detecting module, and an electronic apparatus which include the biological information measuring module.

2. Related Art

Hitherto, there have been known measuring modules that are worn around body parts, such as a wrist, by a band or the like and measure biological information such as a wearer's pulse waves, and wristwatch type wrist devices having a function of measuring the biological information. For example, Pamphlet of International Publication No. 2014/091424A2 discloses a biological information measuring apparatus which is worn around the arm (wrist) of a wearer (test subject) and is mounted with a biological information measuring module that measures biological information, such as pulse waves, using an optical detection sensor.

Such a biological information measuring module using an optical detecting sensor (optical sensor) and the biological information measuring apparatus using the biological information measuring module optically measure the flow of blood under a skin surface which is a measurement object and convert the measured blood flow into a signal to thereby obtain biological information such as pulse waves, and thus it is important to reduce noise components included in light received by the optical detecting sensor in order to increase measurement accuracy. On the other hand, Pamphlet of International Publication No. 2014/091424A2 proposes that a gap is not provided between a light receiving element and a skin surface which is a measurement object of a wearer (test subject).

In addition, measurement using a light detecting apparatus (optical sensor) is adopted in various fields, and, for example, a spectroscopic sensor is used in order to diagnose and inspect an object. In the light detecting apparatus, there is a concern for a problem caused by a measurement environment such as the erosion of the apparatus which is caused by moisture existing in the measurement environment. In addition, in a type of light detecting apparatus worn on a body, there is also a concern for erosion caused by sweat, and the like. For this reason, it is also considered that the wiring of the light detecting apparatus has to be protected by a silicon nitride film. In this case, light transmittance may deteriorate, and the oxide film (insulating film) is maintained as it is.

Regarding problems of the environmental resistance (or moisture resistance) and light transmittance, in the invention disclosed in JP-A-2014-165191, such problems are solved by using a thin silicon nitride film and silicon oxynitride film. In addition, in the invention disclosed in JP-A-6-21470, a plurality of thin silicon nitride films and silicon oxide films are alternately laminated on each other, thereby solving such problems.

However, in a biological information measuring module and a biological information measuring apparatus using the biological information measuring module disclosed in Pamphlet of International Publication No. 2014/091424A2, a wearing state varies due to on exercise or body motion of a wearer (test subject) who is a user, and thus a gap may be generated between a light receiving element and a skin surface, which results in a difficulty in reducing noise components. Therefore, there have been demands for a biological information measuring module capable of reducing noise components and accurately measuring biological information even in a state where a wearing state varies due to on exercise or body motion of a wearer (test subject) who is a user, and a biological information measuring apparatus using the biological information measuring module.

In addition, in the inventions disclosed in JP-A-2014-165191 and JP-A-6-21470, it is necessary to additionally laminate a particular film such as a silicon oxynitride film, and thus there is a problem in that the throughput and cost of a manufacturing process may deteriorate.

### SUMMARY

An advantage of some aspects of the invention is to solve at least a part of the problems described above, and the invention can be implemented as the following forms or application examples.

### Application Example 1

A biological information measuring module according to this application example includes alight emitting unit that emits light; a light receiving unit that receives the light via an object; and a multilayered film optical filter that is provided on the light receiving unit on a side where the light is incident. The multilayered film optical filter is constituted by a laminated body of five or more layers and 120 or less layers.

According to this application example, since the multilayered film optical filter is constituted by a laminated body of five or more layers and 120 or less layers, it is possible to cut optical components (noise components) which are unnecessary for the measurement of biological information such as, for example, pulse waves and to accurately measure biological information. On the other hand, as the number of layers laminated of the laminated body constituting the multilayered film optical filter increases, the number of formation processes increases, which results in a deterioration in productivity. In addition, when the number of layers laminated exceeds 120, effects (characteristics) as the multilayered film optical filter have few changes. In other words, the effects (characteristics) in a case where the number of layers laminated is 120 are not different from those in a case where the number of layers laminated is equal to or greater than 121, and thus the lamination of 121 or more layers in which the number of formation processes increases is not necessary. Therefore, the number of layers laminated of the laminated body constituting the multilayered film optical filter is set to be equal to or greater than five and equal to or less than 120, and thus it is possible to efficiently cut optical components (noise components) which are unnecessary for the measurement of biological information without degrading productivity, and to provide the biological information measuring module capable of accurately measuring biological information.

### Application Example 2

In the biological information measuring module according to the application example, it is preferable that the multilayered film optical filter is constituted by a laminated body of ten or more layers and 120 or less layers.

According to this application example, the number of layers laminated of the multilayered film optical filter is set to be ten or more, and thus it is possible to further efficiently cut unnecessary optical components (noise components). Specifically, it is possible to reduce the transmittance of light, having an unnecessary wavelength, which serves as a noise component to approximately 2%.

### Application Example 3

In the biological information measuring module according to the application example, it is preferable that the multilayered film optical filter is constituted by a laminated body of twenty or more layers and sixty or less layers.

According to this application example, the number of layers laminated of the multilayered film optical filter is set to be twenty or more, and thus it is possible to further efficiently cut unnecessary optical components (noise components). Specifically, it is possible to reduce the transmittance of light, having an unnecessary wavelength, which serves as a noise component to approximately 0.1%. In addition, the number of layers laminated of the multilayered film optical filter is set to be sixty or less, and thus it is possible to further improve the productivity of the multilayered film optical filter.

### Application Example 4

In the biological information measuring module according to the application example, it is preferable that the multilayered film optical filter is constituted by a laminated body of twenty three or more layers and sixty or less layers.

According to this application example, the number of layers laminated of the multilayered film optical filter is set to be twenty three or more, and thus it is possible to further efficiently cut unnecessary optical components (noise components). Specifically, it is possible to reduce the transmittance of light, having an unnecessary wavelength, which serves as a noise component to approximately 0.05%. Therefore, it is possible to drastically reduce the transmittance of unnecessary optical components (noise components) while improving the productivity of the multilayered film optical filter with sixty or less layers laminated.

### Application Example 5

In the biological information measuring module according to the application example, it is preferable that the multilayered film optical filter is constituted by a laminated body of forty or more layers and sixty or less layers.

According to this application example, the number of layers laminated of the multilayered film optical filter is set to be forty or more, and thus it is possible to further efficiently cut unnecessary optical components (noise components). Specifically, it is possible to rigorously cut light, having a wavelength of 300 nm to 500 nm and having a wavelength of 600 nm to 1000 nm, which serves as a noise component during the measurement of pulse waves. Therefore, it is possible to drastically reduce light having a wavelength of 300 nm to 500 nm and having a wavelength of 600 nm to 1000 nm, which serves as a noise component during the measurement of pulse waves while improving the productivity of the multilayered film optical filter with sixty or less layers laminated.

### Application Example 6

In the biological information measuring module according to the application example, it is preferable that the multilayered film optical filter is constituted by a laminated body of twenty three or more layers and forty or less layers.

According to this application example, it is possible to efficiently cut unnecessary optical components (noise components) and to further improve the productivity of the multilayered film optical filter.

### Application Example 7

In the biological information measuring module according to the application example, it is preferable that a reflective functional layer is provided in at least a portion of a vicinity of the light emitting unit.

According to this application example, light emitted from a peripheral direction of the light emitting unit can be made to be reflected by a reflective functional layer and to be directed to an object. Thereby, it is possible to increase the intensity (light emission intensity) of light directed to the object and to stabilize the measurement accuracy of biological information.

### Application Example 8

In the biological information measuring module according to the application example, it is preferable that a frame is provided between the light emitting unit and the light receiving unit.

According to this application example, it is possible to prevent light emitted from the light emitting unit from directly reaching (being incident on) the light receiving unit by the frame disposed between the light receiving unit and the light emitting unit. Thereby, it is possible to allow light having less noise components to be incident on the light receiving unit and to further improve the measurement accuracy of the biological information measuring module.

### Application Example 9

In the biological information measuring module according to the application example, it is preferable that the frame is formed of a resin or a metal.

According to this application example, the resin or the metal is a material which is easily obtained and processed, and thus it is possible to easily form the frame.

### Application Example 10

In the biological information measuring module according to the application example, it is preferable that the biological information measuring module further includes a supporting unit and the light emitting unit and the light receiving unit are supported by a supporting surface of the supporting unit.

According to this application example, the light emitting unit and the light receiving unit are supported by the supporting surface of the supporting unit, and thus it is possible to achieve space saving and to realize the compact biological information measuring module.

### Application Example 11

In the biological information measuring module according to the application example, it is preferable that a light condensing member is provided on the light emitting unit on a side where the light is emitted.

According to this application example, light emitted from the light emitting unit is condensed by the light condensing member and is directed to an object, and thus it is possible to increase the intensity of the light. Consequently, light incident on the light receiving unit can be intensified, and thus it is possible to accurately perform measurement.

### Application Example 12

In the biological information measuring module according to the application example, it is preferable that a plurality of the light emitting units are provided.

According to this application example, the plurality of light emitting units are provided, and thus it is possible to secure more sufficient light emission intensity. In addition, biological information is detected by detecting light beams from the plurality of light emitting units, and thus it is possible to configure the biological information measuring module having further improved measurement accuracy.

### Application Example 13

In the biological information measuring module according to the application example, it is preferable that a plurality of the light receiving units are provided.

According to this application example, the plurality of light receiving units are provided, and thus it is possible to receive a greater amount of light (light with high intensity) and to configure the biological information measuring module with improved measurement accuracy.

### Application Example 14

In the biological information measuring module according to the application example, it is preferable that the laminated body is configured such that an oxide film and a nitride film are alternately laminated on each other.

According to this application example, the oxide film and the nitride film are alternately laminated on each other, and thus some of incident light changes to transmitted light and reflected light at the boundary between the oxide film and the nitride film. Further, a portion of the reflected light is reflected from the boundary again and is combined with transmitted light. At this time, light having a wavelength that conforms to the length of a light path of the reflected light strengthen due to conformity between phases of the reflected light and the transmitted light, and light having a wavelength that does not conform to the length of a light path of the reflected light weakens due to unconformity between phases of the reflected light and the transmitted light. Thereby, only light having a necessary wavelength can reach the light receiving unit. Therefore, it is possible to configure the biological information measuring module capable of performing measurement more accurately.

### Application Example 15

In the biological information measuring module according to the application example, it is preferable that the lowermost layer of the laminated body on the light receiving unit side is an oxide film.

According to this application example, the oxide film has high adhesiveness with respect to a substrate (for example, a silicon substrate) which is generally used as a base material of the light receiving unit, and thus it is possible to suppress risk such as peeling.

### Application Example 16

In the biological information measuring module according to the application example, it is preferable that the lowermost layer of the laminated body on the light receiving unit side is a nitride film.

According to this application example, since the nitride film has a higher refractive index than that of an oxide film, the lowermost layer located to be close to the light receiving unit is configured as a nitride film, and thus it is possible to more effectively reflect unnecessary light.

### Application Example 17

In the biological information measuring module according to the application example, it is preferable that a resin film is provided on the multilayered film optical filter.

According to this application example, it is possible to increase a waterproofing property and an antifouling property of the multilayered film optical filter by the resin film.

### Application Example 18

A biological information measuring apparatus according to this application example includes the biological information measuring module according to any one of the above-mentioned application examples.

According to this application example, detection (measurement) can be performed more accurately, and the biological information measuring module having a small size and excellent portability is provided, and thus it is possible to stably detect biological information during exercise or the like and to provide the biological information measuring apparatus having a small size and excellent portability (wearability).

### Application Example 19

A light detecting apparatus according to this application example includes a semiconductor substrate; a light detecting element that is formed on a surface of the semiconductor substrate; a wiring that is formed on the semiconductor substrate; and a multilayered film optical filter that is formed on the light detecting element and the wiring. The multilayered film optical filter is formed in such a way that the wiring is positioned inside a region in which the multilayered film optical filter is formed, when seen in a plan view in a direction perpendicular to the semiconductor substrate.

According to this application example, the multilayered film optical filter is formed in such a way that the wiring is positioned inside the region in which the multilayered film optical filter is formed, when seen in a plan view in a direction perpendicular to the semiconductor substrate, thereby preventing the wiring from getting wet by water. Accordingly, it is possible to improve moisture resistance and light transmittance while suppressing a manufacturing cost.

### Application Example 20

In the light detecting apparatus according to the application example, the light detecting apparatus may include a pad for external connection of the light detecting apparatus, and the multilayered film optical filter may be formed so as to have an opening at a position of the pad, when seen in the plan view.

According to this application example, it is possible to prevent the multilayered film optical filter from closing the pad.

### Application Example 21

In the light detecting apparatus according to the application example, the pad may be a pad for connecting an end of the light detecting element to the outside.

According to this application example, it is possible to electrically connect an end of the light detecting element to the outside.

### Application Example 22

In the light detecting apparatus according to the application example, the light detecting apparatus may further include a light shielding layer that is formed on the semiconductor substrate, and the multilayered film optical filter may be formed in such a way that the light shielding layer is positioned inside a region in which the multilayered film optical filter is formed, when seen in the plan view.

According to this application example, it is possible to shield light in a region in which the light detecting element is not formed.

### Application Example 23

In the light detecting apparatus according to the application example, the multilayered film optical filter may be formed in such a way that one side of the multilayered film optical filter is positioned between one side of the light detecting apparatus and one side of the light shielding layer which faces the one side of the light detecting apparatus, when seen in the plan view.

According to this application example, the multilayered film optical filter can be formed so as to cover the light shielding layer.

### Application Example 24

In the light detecting apparatus according to the application example, the wiring is a wiring for electrically connecting the pad and an end of the light detecting element.

According to this application example, it is possible to electrically connect an external device and the light detecting element.

### Application Example 25

In the light detecting apparatus according to the application example, the light detecting apparatus may further include an insulating layer. When a direction when seen in the plan view is set to be a first direction and a direction perpendicular to the first direction is set to be a second direction, the multilayered film optical filter may be formed on the second direction side of the insulating layer in an end of the insulating layer.

According to this application example, it is possible to prevent water from entering the inside of the light detecting apparatus from the side surface of the apparatus.

### Application Example 26

In the light detecting apparatus according to the application example, the multilayered film optical filter is formed so as to cover the wiring and the light detecting element when seen in the plan view.

According to this application example, the multilayered film optical filter can be formed in such a way that the wiring is positioned inside a region in which the multilayered film optical filter is formed, when seen in a plan view in a direction perpendicular to the semiconductor substrate.

### Application Example 27

A light detecting apparatus according to this application example includes a semiconductor substrate; a light detecting element that is formed on a surface of the semiconductor substrate; alight shielding layer that is formed on the semiconductor substrate; and a multilayered film optical filter that is formed on the light detecting element and the light shielding layer. The multilayered film optical filter is formed in such a way that one side of the multilayered film optical filter is positioned between one side of the light detecting apparatus and one side of the light shielding layer which faces the one side of the light detecting apparatus, when seen in a plan view in a direction perpendicular to the semiconductor substrate.

According to this application example, the multilayered film optical filter can be formed so as to cover the light shielding layer. When the light shielding layer is a metal layer, it is possible to prevent corrosion caused by water.

### Application Example 28

A light detecting module according to this application example includes the light detecting apparatus according to any one of the above-mentioned application examples.

### Application Example 29

An electronic apparatus according to this application example includes the light detecting apparatus according to any one of the above-mentioned application examples.

### BRIEF DESCRIPTION OF THE DRAWINGS

The invention will be described with reference to the accompanying drawings, wherein like numbers reference like elements.

FIG. 1 is a perspective view illustrating the exterior of a biological information measuring apparatus according to a first embodiment.

FIG. 2 is a perspective view illustrating the exterior of the biological information measuring apparatus according to the first embodiment when seen from a diagonally upper side.

FIG. 3 is a side view illustrating the exterior of the biological information measuring apparatus according to the first embodiment.

FIG. 4 is a diagram illustrating the wearing of the biological information measuring apparatus and communication with a terminal device.

FIG. 5 is a functional block diagram of the biological information measuring apparatus.

FIG. 6 is a plan view illustrating Configuration Example 1 of a sensor unit as a biological information measuring module.

FIG. 7 is a front cross-sectional view illustrating Configuration Example 1 of the sensor unit.

FIG. 8 is a partially enlarged view (front cross-sectional view) of FIG. 7 illustrating an example of a light receiving unit in Configuration Example 1 of the sensor unit.

FIG. 9 is a partially enlarged view (front cross-sectional view) of FIG. 7 illustrating an example of the light receiving unit in Configuration Example 1 of the sensor unit.

FIG. 10A is a graph illustrating a characteristic example of a multilayered film optical filter of a structure in which four layers are laminated.

FIG. 10B is a graph illustrating a characteristic example of a multilayered film optical filter of a structure in which five or more layers are laminated.

FIG. 11A is a schematic plan view illustrating a configuration of a light detecting apparatus according to Example 1.

FIG. 11B is a cross-sectional view taken along line X-X′ of FIG. 11A.

FIG. 11C is a partially enlarged cross-sectional view of a portion of FIG. 11B.

FIG. 12A is a schematic plan view illustrating a modification example of a configuration of the light detecting apparatus of Example 1.

FIG. 12B is a cross-sectional view taken along line X-X′ of FIG. 12A.

FIG. 12C is a partially enlarged cross-sectional view of a portion of FIG. 12B.

FIG. 13A is a schematic plan view illustrating a comparative example of a configuration of a light detecting apparatus.

FIG. 13B is a cross-sectional view taken along line X-X′ of FIG. 13A.

FIG. 13C is a partially enlarged cross-sectional view of a portion of FIG. 13B.

FIG. 14A is a schematic plan view illustrating a configuration of a light detecting apparatus according to Example 2.

FIG. 14B is a cross-sectional view taken along line X-X′ of FIG. 14A.

FIG. 14C is a partially enlarged cross-sectional view of a portion of FIG. 14B.

FIG. 15 is a plan view illustrating Configuration Example 2 of a sensor unit as the biological information measuring module according to the first embodiment.

FIG. 16 is a plan view illustrating Configuration Example 3 of a sensor unit as the biological information measuring module according to the first embodiment.

FIG. 17 is a plan view illustrating Configuration Example 4 of a sensor unit as a biological information measuring module.

FIG. 18 is a cross-sectional view illustrating an example of the art of a biological information measuring apparatus according to a second embodiment.

FIG. 19 is a perspective view illustrating the biological information measuring apparatus according to the second embodiment.

FIG. 20 is a front view illustrating a biological information measuring apparatus according to a third embodiment.

FIG. 21 is a perspective view illustrating a biological information measuring apparatus according to a fourth embodiment.

FIG. 22 is a cross-sectional view illustrating a biological information measuring apparatus according to a fifth embodiment.

FIG. 23 is a flow chart illustrating a method of manufacturing the biological information measuring apparatus according to the second to fifth embodiments.

FIG. 24 is a schematic diagram illustrating a web page serving as a starting point of a health manager in a biological information measuring apparatus according to a sixth embodiment.

FIG. 25 is a diagram illustrating an example of a nutrition web page.

FIG. 26 is a diagram illustrating an example of an activity level web page.

FIG. 27 is a diagram illustrating an example of a mental concentration web page.

FIG. 28 is a diagram illustrating an example of a sleep web page.

FIG. 29 is a diagram illustrating an example of a daily activity web page.

FIG. 30 is a diagram illustrating an example of a health degree web page.

FIG. 31 is a partial cross-sectional view illustrating a modification example of a light receiving unit.

FIG. 32 is a partial cross-sectional view illustrating a modification example of a light emitting unit.

### DESCRIPTION OF EXEMPLARY EMBODIMENTS

Hereinafter, this embodiment will be described. Meanwhile, this embodiment described below does not improperly limit the contents of the invention which are described in the appended claims. In addition, all of the components described in this embodiment are not necessarily essential components of the invention.

### First Embodiment

### 1. Overall Configuration Example of Biological Information Measuring Apparatus

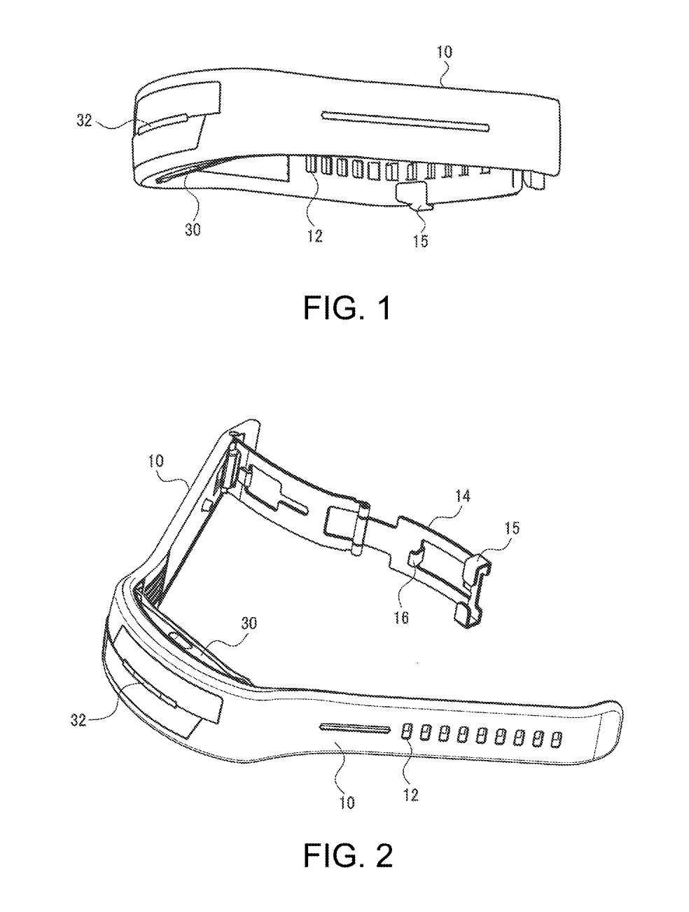

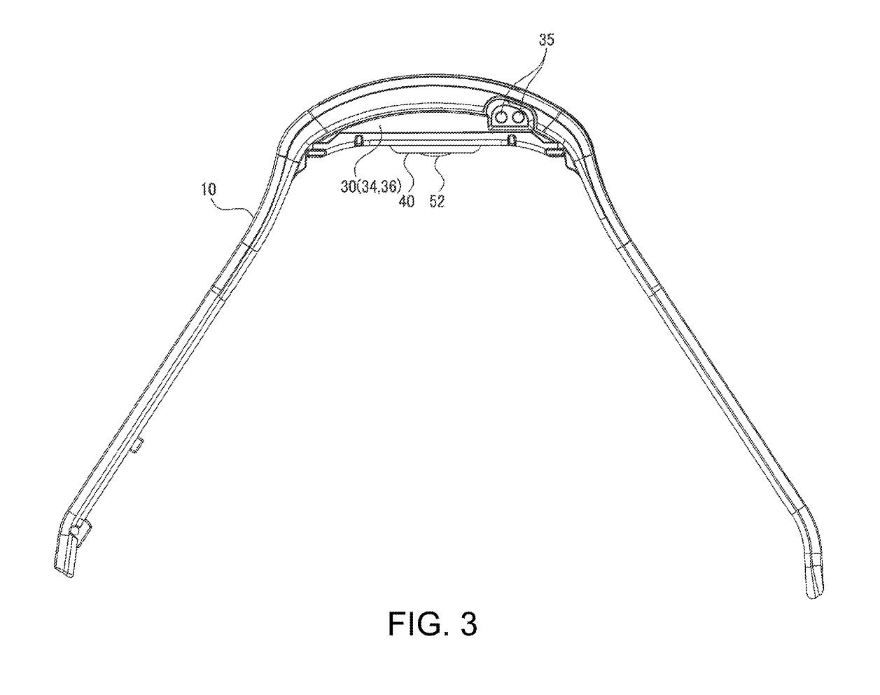

FIGS. 1 to 3 are schematic diagrams illustrating the exterior of a biological information measuring apparatus (biological information detecting apparatus) according to a first embodiment. FIG. 1 is a diagram when the biological information measuring apparatus is seen from the front, FIG. 2 is a diagram when the biological information measuring apparatus of FIG. 1 is obliquely seen from above, and FIG. 3 is a diagram when the biological information measuring apparatus is seen from the side.

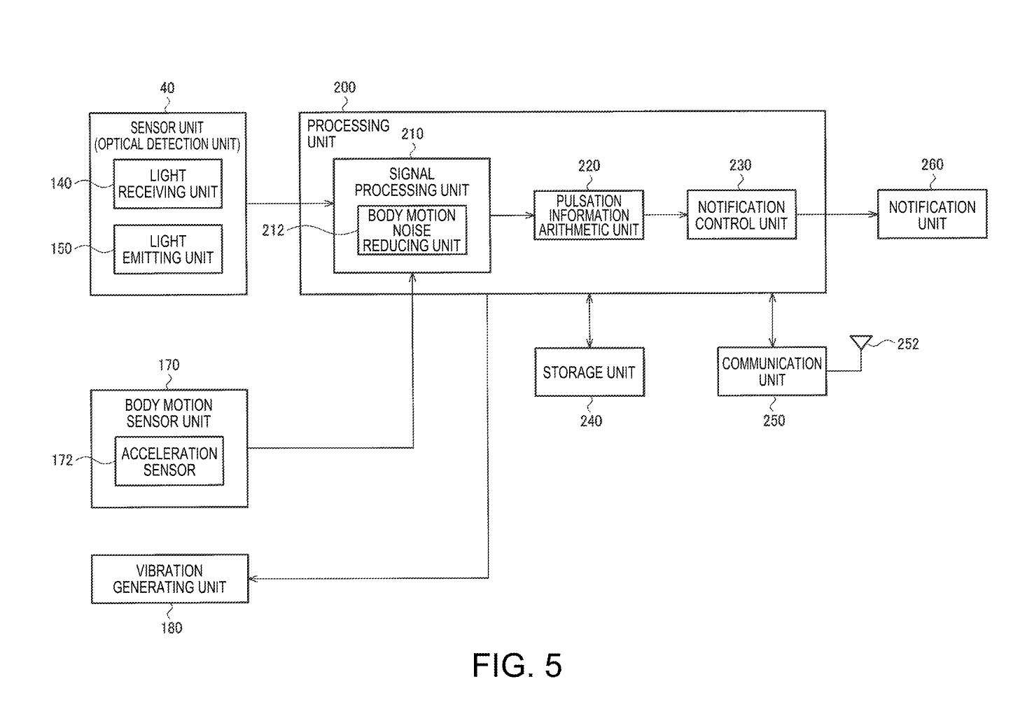

As illustrated in FIGS. 1 to 3, the biological information measuring apparatus of this embodiment includes a band portion 10, a case portion 30, and a sensor unit 40 as a biological information measuring module. The case portion 30 is attached to the band portion 10. The sensor unit 40 is provided in the case portion 30. In addition, the biological information measuring apparatus includes a processing unit 200 as illustrated in FIG. 5 to be described later. The processing unit 200 is provided in the case portion 30, and detects biological information on the basis of a detection signal from the sensor unit 40. Meanwhile, the biological information measuring apparatus of this embodiment is not limited to the configurations illustrated in FIGS. 1 to 3, and various modifications such as the omission of some of the components thereof, replacement with other components, or the addition of other components can be made.

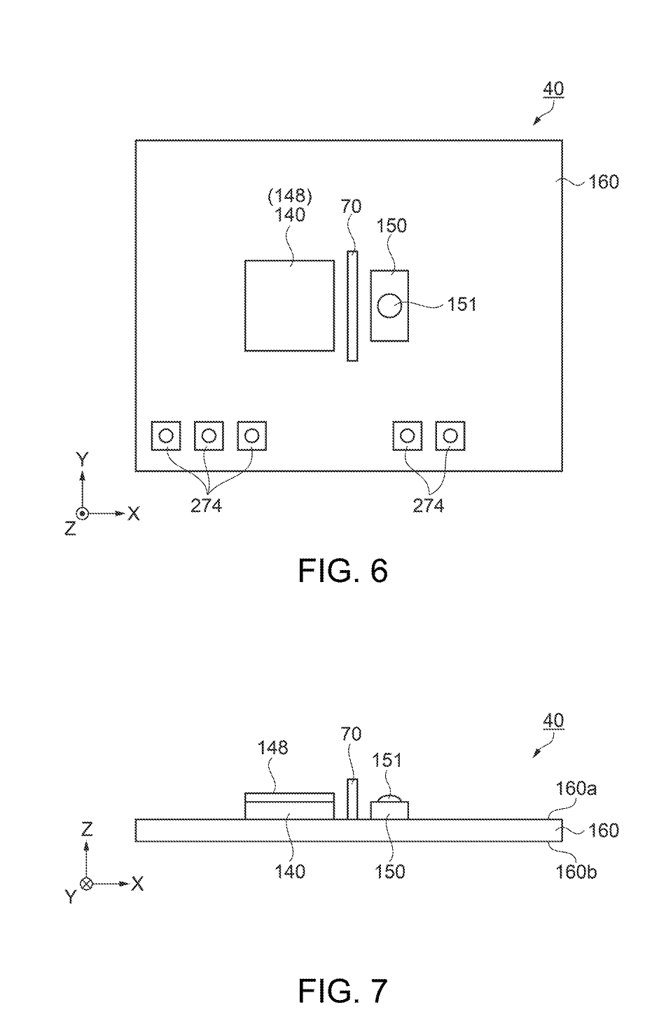

The sensor unit 40 as a biological information measuring module includes a substrate 160, a light emitting unit 150, a light receiving unit 140, a wall portion 70 as a frame, and other members, as described later with reference to FIGS. 6 and 7. A component including these components can be configured as a light detection unit (not shown). Meanwhile, the other members in Configuration Example 1 include a convex portion, a pressing suppressing portion, and the like which are realized by a light transmitting member. A modification can also be made in which the light detection unit according to this embodiment includes these members, that is, the entire sensor unit 40 corresponds to the light detection unit.

Referring back to FIGS. 1 to 3, the band portion 10 is wound around the wrist of a wearer (hereinafter, also referred to as a user) so that the biological information measuring apparatus is worn thereon. The band portion 10 includes band holes 12 and a buckle portion 14. The buckle portion 14 includes a band insertion portion 15 and a protrusion portion 16. The user inserts one end side of the band portion 10 into the band insertion portion 15 of the buckle portion 14 and inserts the protrusion portion 16 of the buckle portion 14 into the band hole 12 of the band portion 10 to thereby wear the biological information measuring apparatus around his or her wrist. In this case, the magnitude of pressing (pressing against the surface of the wrist) by the sensor unit 40 to be described later is adjusted according to into which of the band holes 12 the protrusion portion 16 is inserted.

The case portion 30 is equivalent to a main body portion of the biological information measuring apparatus. Various components of the biological information measuring apparatus such as the sensor unit 40 and the processing unit 200 (see FIG. 5) are provided within the case portion 30. That is, the case portion 30 is a housing that accommodates the components. The case portion 30 includes, for example, a top case 34 which is positioned on the opposite side to the wrist and a bottom case 36 which is positioned on the wrist side. Meanwhile, the case portion 30 may not be configured so as to separate into the top case 34 and the bottom case 36.

The case portion 30 is provided with a light emitting window portion 32. The light emitting window portion 32 is formed of a light transmitting member. In addition, the case portion 30 is provided with a light emitting unit (LED, alight emitting unit for a notice which is different from the light emitting unit 150 of the light detection unit) which is mounted on a flexible substrate, and light from the light emitting unit is emitted to the outside of the case portion 30 through the light emitting window portion 32.

As illustrated in FIG. 3, the case portion 30 is provided with a terminal portion 35. When the biological information measuring apparatus is mounted on a cradle not shown in the drawing, a terminal portion of the cradle and the terminal portion 35 of the case portion 30 are electrically connected to each other. Thereby, a secondary battery (battery) provided in the case portion 30 can be charged.

The sensor unit 40 as a biological information measuring module detects biological information such as pulse waves of a test subject. For example, the sensor unit 40 includes a light receiving unit 140 and a light emitting unit 150 as illustrated in FIGS. 5 to 7 to be described later. In addition, the sensor unit 40 is formed of the light transmitting member and includes the convex portion 52 that comes into contact with a test subject's skin surface and applies pressure. In this manner, the light emitting unit 150 emits light in a state where the convex portion 52 applies pressure to the skin surface, the light receiving unit 140 receives the light reflected by the test subject (blood vessel), and the light reception result thereof is output to the processing unit 200 as a detection signal. In addition, the processing unit 200 detects biological information, such as pulse waves, on the basis of the detection signal from the sensor unit 40. Meanwhile, biological information to be detected by the biological information measuring apparatus of this embodiment is not limited to pulse waves (pulse rate), and the biological information measuring apparatus may be an apparatus that detects biological information (for example, oxygen saturation in the blood, body temperature, heartbeat, and the like) other than pulse waves.

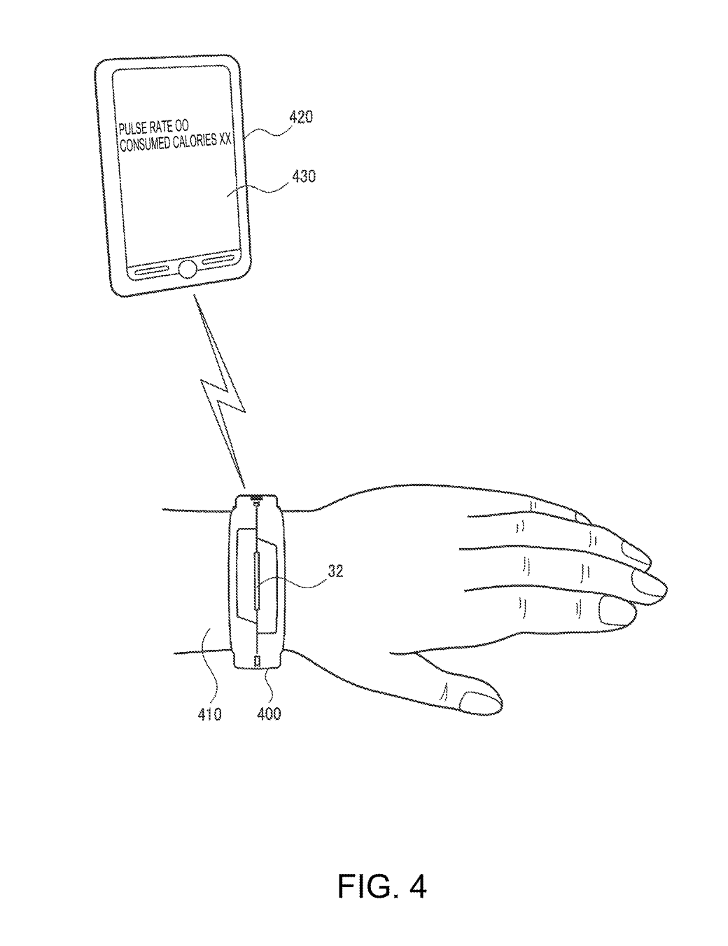

FIG. 4 is a schematic diagram illustrating the wearing of a biological information measuring apparatus 400 and communication with a terminal device 420. As illustrated in FIG. 4, a user who is a test subject wears the biological information measuring apparatus 400 around a wrist 410 like a wristwatch. As illustrated in FIG. 3, the sensor unit 40 is provided on a surface of the case portion 30 on the test subject side. Accordingly, when the biological information measuring apparatus 400 is worn, the convex portion 52 of the sensor unit 40 comes into contact with the skin surface of the wrist 410 and applies pressure. In this state, the light emitting unit 150 of the sensor unit 40 emits light, and the light receiving unit 140 receives the reflected light, and thus biological information such as pulse waves is detected.

The biological information measuring apparatus 400 and the terminal device 420 are connected to each other for communication, and thus data can be exchanged therebetween. The terminal device 420 is a portable communication terminal such as a smartphone, a mobile phone, or a feature phone. Alternatively, the terminal device 420 may be an information processing terminal such as a tablet computer. Proximity wireless communication such as Bluetooth (registered trademark) can be adopted as a communication connection between the biological information measuring apparatus 400 and the terminal device 420. In this manner, the biological information measuring apparatus 400 and the terminal device 420 are connected to each other for communication connection, and thus various pieces of information such as a pulse rate and consumed calories can be displayed on a display unit 430 (LCD or the like) of the terminal device 420. That is, various pieces of information obtained on the basis of the detection signal of the sensor unit 40 can be displayed. Meanwhile, the arithmetic processing of information such as a pulse rate or consumed calories may be performed by the biological information measuring apparatus 400, or at least a portion thereof may be performed by the terminal device 420.

The biological information measuring apparatus 400 is provided with the light emitting window portion 32, so that a user is notified of various pieces of information by light emission (lighting, blinking) of a light emitting body for a notice (not shown). For example, in the case of entering a fat combustion zone in information such as consumed calories or in the case of leaving the fat combustion zone, this is given notice of by the light emission of the light emitting body through the light emitting window portion 32. In addition, when an e-mail is received in the terminal device 420, the biological information measuring apparatus 400 is notified of the received e-mail from the terminal device 420. The light emitting body of the biological information measuring apparatus 400 emits light, and thus a user is notified of the reception of an e-mail or the like.

In this manner, in the example illustrated in FIG. 4, the biological information measuring apparatus 400 is not provided with a display unit such as an LCD, and thus information required to be given notice of by characters or numerals is displayed on the display unit 430 of the terminal device 420. In this manner, in the example illustrated in FIG. 4, a user is notified of the necessary minimum information by the light emission of the light emitting body without providing a display unit such as an LCD, thereby realizing a reduction in the size of the biological information measuring apparatus 400. In addition, the biological information measuring apparatus 400 is not provided with a display unit, and thus it is possible to improve the beauty of the biological information measuring apparatus 400.

FIG. 5 is a functional block diagram of the biological information measuring apparatus of this embodiment. The biological information measuring apparatus illustrated in FIG. 5 includes the sensor unit 40 as a biological information measuring module, a body motion sensor unit 170, a vibration generating unit 180, the processing unit 200, a storage unit 240, a communication unit 250, an antenna 252, and a notification unit 260. Meanwhile, the biological information measuring apparatus of this embodiment is not limited to the configuration illustrated in FIG. 5, and various modifications such as the omission of some of the components thereof, replacement with other components, or the addition of other components can be made.

The sensor unit 40 as a biological information measuring module detects biological information such as pulse waves, and includes the light receiving unit 140 and the light emitting unit 150. A pulse wave sensor (photoelectric sensor) is realized by the light receiving unit 140, the light emitting unit 150, and the like. The sensor unit 40 outputs a signal detected by the pulse wave sensor as a pulse wave detection signal.

The body motion sensor unit 170 outputs a body motion detection signal which is a signal varying in response to body motion, on the basis of pieces of sensor information of various sensors. The body motion sensor unit 170 includes, for example, an acceleration sensor 172 as a body motion sensor. Meanwhile, the body motion sensor unit 170 may include a pressure sensor, a gyro sensor, or the like as the body motion sensor.

The processing unit 200 performs various types of signal processes and control processes, for example, with the storage unit 240 as a work area, and can be realized by, for example, a processor such as a CPU or a logic circuit such as an ASIC. The processing unit 200 includes a signal processing unit 210, a pulsation information arithmetic unit 220, and a notification control unit 230.

The signal processing unit 210 performs various types of signal processes (filtering and the like), and performs signal processing on, for example, a pulse wave detection signal from the sensor unit 40, a body motion detection signal from the body motion sensor unit 170, or the like. For example, the signal processing unit 210 includes a body motion noise reducing unit 212. The body motion noise reducing unit 212 performs processing for reducing (removing) body motion noise which is noise caused by body motion, from the pulse wave detection signal, on the basis of the body motion detection signal from the body motion sensor unit 170. Specifically, the body motion noise reducing unit performs a noise reduction process using, for example, an adaptive filter.

The pulsation information arithmetic unit 220 performs arithmetic processing of pulsation information on the basis of a signal from the signal processing unit 210, and the like. The pulsation information is information such as a pulse rate. Specifically, the pulsation information arithmetic unit 220 obtains a spectrum by performing frequency analysis processing such as FFT on the pulse wave detection signal having been subjected to the noise reduction process by the body motion noise reducing unit 212, and performs a process of setting a representative frequency in the obtained spectrum as a frequency of a heartbeat. A value obtained by increasing the obtained frequency by 60 times is set to be a pulse rate (heart rate) which is generally used. Meanwhile, the pulsation information is not limited to the pulse rate itself, and may be various other pieces of information (for example, the frequency or cycle of a heartbeat) which indicate, for example, a pulse rate. In addition, the pulsation information may be information indicating the state of pulsation, or a value indicating, for example, the amount of blood itself may be set as pulsation information.

The notification control unit 230 controls the notification unit 260. The notification unit 260 (notification device) notifies a user of various pieces of information under the control of the notification control unit 230. For example, a light emitting body for a notice can be used as the notification unit 260. In this case, the notification control unit 230 controls a current flowing to an LED to thereby control the lighting, blinking, or the like of the light emitting body. Meanwhile, the notification unit 260 may be a display unit, such as an LCD, a buzzer, or the like.

In addition, the notification control unit 230 controls the vibration generating unit 180. The vibration generating unit 180 notifies a user of various pieces of information by vibration. The vibration generating unit 180 can be realized by, for example, a vibration motor (vibrator). The vibration motor generates vibration, for example, by rotating an eccentric weight. Specifically, the eccentric weight is attached to both ends of a driving shaft (rotor shaft) so that the motor itself shakes. The vibration of the vibration generating unit 180 is controlled by the notification control unit 230. Meanwhile, the vibration generating unit 180 is not limited to such a vibration motor, and various modifications can be made. The vibration generating unit 180 may be realized by, for example, a piezo element.

For example, a notice of start-up at the time of power-on, a notice of the first success in detecting pulse waves, a warning when a pulse-wave undetectable state is continued for a fixed period of time, a notice at the time of the movement of a fat combustion zone, a warning at the time of a battery voltage drop, a notice of a wake-up alarm, or a notice of an e-mail or a call from a terminal device such as a smartphone can be performed by the vibration of the vibration generating unit 180. Meanwhile, the pieces of information may be given notice of by a light emitting unit for a notice, or may be given notice of by both the vibration generating unit 180 and the light emitting unit.

The communication unit 250 performs communication with the external terminal device 420 as described in FIG. 4. For example, the communication unit performs wireless communication according to a standard such as Bluetooth (registered trademark). Specifically, the communication unit 250 receives a signal from the antenna 252 and transmits a signal to the antenna 252. The function of the communication unit 250 can be realized by a processor for communication or a logic circuit such as an ASIC.

### 2. Configuration Example of Sensor Unit as Biological Information Measuring Module

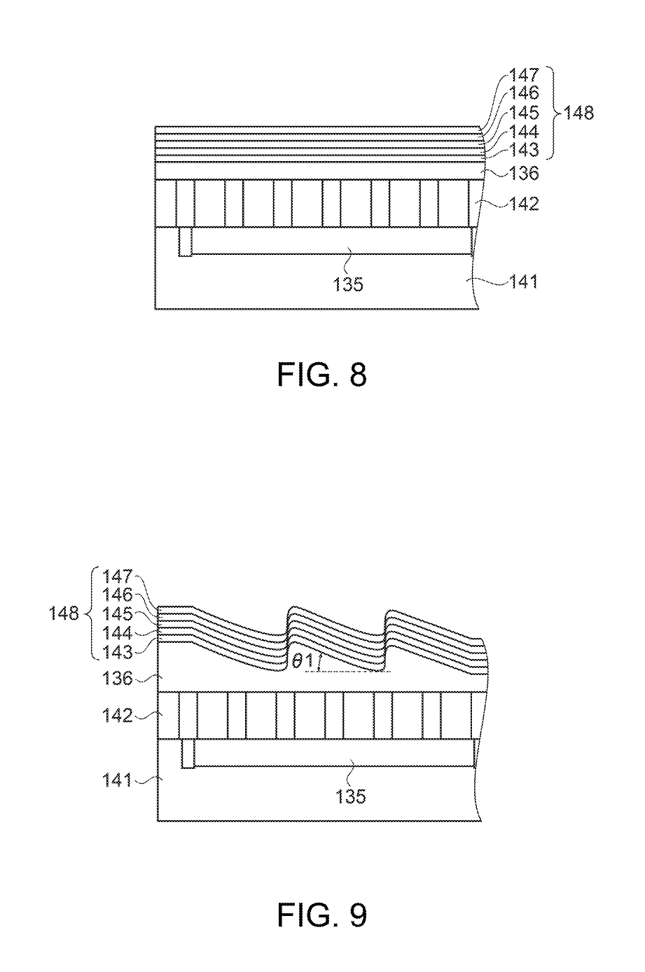

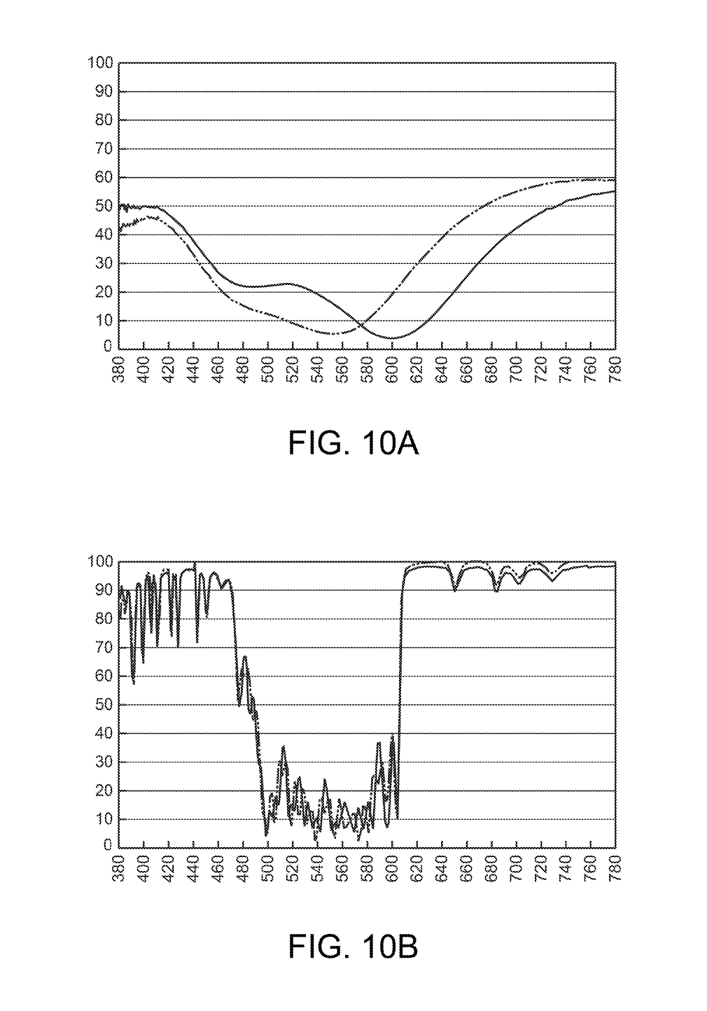

A detailed configuration example of the sensor unit 40 as a biological information measuring module will be described with reference to FIGS. 6 to 9 and FIGS. 10A and 10B. FIG. 6 is a plan view illustrating Configuration Example 1 of the sensor unit 40, and FIG. 7 is a front cross-sectional view. FIG. 8 is a partially enlarged view (front cross-sectional view) of FIG. 7 illustrating a configuration example of the light receiving unit of the sensor unit in Configuration Example 1, and FIG. 9 illustrates a configuration example different from that of FIG. 8. FIG. 7 illustrates characteristics of a multilayered film optical filter provided in a light receiving unit constituting a sensor unit, FIG. 10A is a graph illustrating a characteristic example of a structure in which four layers are laminated, and FIG. 10B is a graph illustrating a characteristic example of a structure in which five or more layers are laminated.

### Configuration Example 1 of Sensor Unit

First, Configuration Example 1 of the sensor unit 40 will be described with reference to FIGS. 6 to 9. The sensor unit 40 of Configuration Example 1 includes a light receiving unit 140, a light emitting unit 150, and a wall portion 70 as a frame which is provided between the light receiving unit 140 and the light emitting unit 150. The light receiving unit 140 and the light emitting unit 150 are lined up at a predetermined interval, and are mounted on a supporting surface 160a of a substrate 160 (sensor substrate) as a supporting unit. The light emitting unit 150 emits light to an object (test subject or the like). The light receiving unit 140 receives light (reflected light, transmitted light, or the like) via the object. For example, when the light emitting unit 150 emits light and the light is reflected by an object (for example, a blood vessel), the light receiving unit 140 receives and detects the reflected light. The light receiving unit 140 can be realized by a light receiving element such as a photodiode. The light emitting unit 150 can be realized by a light emitting element such as an LED. For example, the light receiving unit 140 can be realized by a diode element of a PN junction which is formed on a semiconductor substrate 141, or the like. In this case, an angle limiting filter 142 for narrowing a light reception angle to be described later or a multilayered film optical filter 148 functioning as a wavelength limiting filter that limits a wavelength of light incident on a light receiving element may be formed on the diode element.

Meanwhile, a dome-type lens 151 (condensing lens in a broad sense) as a light condensing member which is provided in the light emitting unit 150 is a lens for condensing light from an LED chip (light emitting element chip in a broad sense) which is resin-sealed (sealed with a light transmitting resin) in the light emitting unit 150. That is, in the light emitting unit 150 which is a surface-mounted type, the LED chip is disposed below the dome-type lens 151, and light from the LED chip is condensed by the dome-type lens 151 and is emitted to an object. Thereby, the intensity of light with which an object is irradiated can be increased, and thus it is possible to improve the optical efficiency of the light detection unit (sensor unit 40) and to perform measurement more accurately.

In addition, as illustrated in FIGS. 8 and 9, the light receiving unit 140 is configured to include a photodiode element 135 of a PN junction which is formed on the semiconductor substrate 141, the angle limiting filter 142 for narrowing a light reception angle, a protection layer 136 (angle structure), the multilayered film optical filter 148 functioning as a wavelength limiting filter that limits a wavelength of light incident on the light receiving unit 140, and the like.

The photodiode element 135 is formed on the semiconductor substrate 141. The photodiode element 135 is formed by an impurity region formed therein by ion implantation or the like. For example, the photodiode element 135 is realized by a PN junction between a P-substrate and an N-type impurity region formed on the P-substrate. Alternatively, the photodiode element 135 is realized by a PN junction between a deep N-well and a P-type impurity region formed on the deep N-well (N-type impurity region).

The angle limiting filter 142 is formed of a light shielding material (light absorbing material or a light reflecting material) having a light shielding property with respect to a wavelength detected by the photodiode element 135. Specifically, the angle limiting filter 142 is formed by a wiring forming step of a semiconductor process, and is constituted by, for example, a conductive layer such as an aluminum (light reflecting material) wiring layer and a conductive plug such as a tungsten (light absorbing material) plug. An aspect ratio of the length of the base (for example, the longest diagonal line of the bottom face, or a major axis) of the angle limiting filter 142 to the height thereof is set in accordance with a transmission wavelength band of the multilayered film optical filter 148. An opening portion (hollow portion) of the angle limiting filter 142 is formed of a transparent material with respect to a wavelength detected by the photodiode element 135, and is formed (filled) of an insulating layer such as SiO₂ (silicon oxide film).

The angle limiting filter 142 can be formed by a wiring layer forming step of another circuit (not shown) formed on the semiconductor substrate 141. Specifically, the angle limiting filter 142 is formed simultaneously with the formation of a wiring layer of the circuit, and is formed by the entirety or a portion of the wiring layer forming step. For example, the angle limiting filter 142 is formed by forming an aluminum (in a broad sense, a light reflecting material) wiring layer through aluminum sputtering, forming an insulating film through SiO₂ deposition, or forming a contact through tungsten (in a broad sense, a light absorbing material) deposition. Meanwhile, the angle limiting filter 142 is not limited to the aluminum (light reflecting material) wiring layer and the tungsten (light absorbing material) contact, and may be formed of a wiring layer including a light absorbing material, such as tungsten, or a contact including a light reflecting material such as aluminum. Here, as the angle limiting filter is formed of a light absorbing material, a light shielding property increases.

The protection layer 136 is formed on the angle limiting filter 142. The protection layer 136 may be flat as illustrated in FIG. 8, or may have an inclined surface having an inclination angle varying depending on a transmission wavelength of the multilayered film optical filter 148 as illustrated in FIG. 9. Specifically, a plurality of inclined surfaces having an inclination angle θ1 with respect to a plane (upper surface) of the semiconductor substrate 141 are formed on the photodiode element 135. The protection layer 136 is formed by processing an insulating film such as SiO₂ by etching, a CMP, a gray scale lithography technique, or the like.

The multilayered film optical filter 148 is also referred to as an optical bandpass filter, and is formed by a laminated body (multi-layered thin film), including the protection layer 136, which is laminated on the upper side of the semiconductor substrate 141. A transmission wavelength band of the multilayered film optical filter 148 is determined by the thickness of the laminated body, the inclination angle θ1 of the protection layer 136, an incident light limiting angle (aspect ratio) of the angle limiting filter 142, and the like. The multilayered film optical filter 148 is configured such that the transmission wavelength thereof varies depending on an inclination angle, and thus is laminated by the same multi-layered film forming step instead of being laminated separate steps for individual transmission wavelengths.

The multilayered film optical filter 148 is formed by a laminated body constituted by a plurality of sets of multi-layered thin films having different transmission wavelengths. For example, as illustrated in FIG. 8, the protection layer 136 which does not have an inclination angle or has an extremely small inclination angle, that is, which is flat may be provided on the angle limiting filter 142, and a flat laminated body (multi-layered thin film) may be formed on the protection layer 136. The plurality of sets of multi-layered thin films are formed in such a way that the films are laminated one layer by one layer by a thin film forming step.

For example, as illustrated in FIG. 9, the multilayered film optical filter 148 may be formed of a laminated body constituted by a multi-layered thin film that is inclined at an angle θ1 depending on a transmission wavelength with respect to the semiconductor substrate 141. More specifically, the multilayered film optical filter 148 is formed of a laminated body constituted by a plurality of sets of multi-layered thin films having different transmission wavelengths. The plurality of sets of multi-layered thin films have an inclination angle θ1 varying depending on a transmission wavelength with respect to the semiconductor substrate 141, and are formed in a thin film forming step at the same time. For example, as illustrated in FIG. 9, the plurality of multi-layered thin films having an inclination angle θ1 are contiguously arrayed, thereby forming a set of multi-layered thin films. Meanwhile, when multi-layered thin films having a different inclination angle θn are contiguously disposed and the multi-layered thin films having the inclination angle θn are repeatedly disposed, a set of multi-layered thin films (laminated bodies) may be formed by a plurality of multi-layered thin films having the same inclination angle (for example, 01).

The laminated body of the multilayered film optical filter 148 which is constituted by multi-layered thin films is configured such that a silicon oxide (SiO₂) film 143 as a first oxide film is provided as a first layer, a silicon nitride (Si₃N₄) film 144 as a first nitride film is provided as a second layer, a silicon oxide (SiO₂) film 145 as a second oxide film is provided as a third layer, a silicon nitride (Si₃N₄) film 146 as a second nitride film is provided as a fourth layer, and a silicon oxide (SiO₂) film 147 as a third oxide film is provided as a fifth layer from the protection layer 136 side, and subsequently, the films are alternately laminated on each other in this order. In this manner, the oxide film (silicon oxide (SiO₂) film) and the nitride film (silicon nitride (Si₃N₄) film) are alternately laminated on each other, and thus some of incident light changes to transmitted light and reflected light at the boundary between the oxide film and the nitride film. Further, a portion of the reflected light is reflected from the boundary and is combined with transmitted light. At this time, light having a wavelength that conforms to the length of a light path of the reflected light strengthens due to conformity between phases of the reflected light and the transmitted light, and light having a wavelength that does not conform to the length of a light path of the reflected light weakens due to unconformity between phases of the reflected light and the transmitted light. Thereby, only light having a necessary wavelength can reach the photodiode element 135 that receives light. Therefore, measurement can be performed more accurately.

Meanwhile, in the drawing, a five-layered laminated body is shown as an example, but the invention is not limited thereto. It is preferable that the laminated body is constituted by a multi-layered thin film of five or more layers and 120 or less layers. When the laminated body is constituted by five or more layers, the laminated body can gradually improve a function as a wavelength limiting filter. On the other hand, as the number of layers laminated increases, the number of formation processes increases, which results in a deterioration in productivity. Here, even when the number of layers laminated exceeds 120, effects (characteristics) as the wavelength limiting filter have few changes. In other words, the effects (characteristics) in a case where the number of layers laminated is 120 are not different from those in a case where the number of layers laminated is equal to or greater than 121, and thus it may be said that the lamination of 121 or more layers in which the number of formation processes increases is not necessary. Therefore, the number of layers laminated of the laminated body which constitutes the multilayered film optical filter 148 is set to be equal to or greater than five and equal to or less than 120, and thus it is possible to efficiently cut optical components (noise components) which are unnecessary for the measurement of biological information without extremely degrading productivity.

Here, reference will be made to graphs illustrated in FIGS. 10A and 10B to describe a correlation between the number of multi-layered thin films (the number of films laminated) in the laminated body of the multilayered film optical filter 148 and a light shielding rate of light, having a wavelength of 300 nm to 500 nm and having a wavelength of 600 nm to 1000 nm, which serves as a noise component during the measurement of pulse waves. FIG. 10A illustrates a characteristic example (two samples) of a configuration of a wavelength limiting filter having a structure in which four layers are laminated, and FIG. 10B illustrates a characteristic example (two samples) in a case where a wavelength limiting filter has a structure in which five layers are laminated. Meanwhile, in the drawings, a horizontal axis represents a wavelength (nm) of light, and a vertical axis represents a measured value of a light shielding rate in each wavelength. As illustrated in FIG. 10A, in the wavelength limiting filter having a structure in which four layers are laminated, the light shielding rate of light, having a wavelength of 380 nm to 500 nm in a range of 300 nm to 500 nm and having a wavelength of 600 nm to 780 nm in a range of 600 nm to 1000 nm, which serves as a noise component during the measurement of pulse waves is approximately 40% to 60%, and thus it can be understood that approximately half of the light having a wavelength in this range is transmitted. Meanwhile, although not shown in the drawings, the light shows the same behavior also in ranges of 380 nm to 300 nm and 780 nm to 1000 nm. On the other hand, the laminated body is configured to have a five-layered structure, and thus the light shielding rate of light, having a wavelength of 380 nm to 500 nm in a range of 300 nm to 500 nm and having a wavelength of 600 nm to 780 nm in a range of 600 nm to 1000 nm, which serves as a noise component during the measurement of pulse waves can be set to be equal to or greater than 80%, as illustrated in FIG. 10B. Meanwhile, although not shown in the drawings, the light shows the same behavior also in ranges of 380 nm to 300 nm and 780 nm to 1000 nm. In this manner, the laminated body is configured to have a five-layered structure, and thus it is possible to more efficiently and rigorously cut (shield) light, having a wavelength of 300 nm to 500 nm and having a wavelength of 600 nm to 1000 nm, which serves as a noise component during the measurement of pulse waves.

Referring back to FIGS. 6 to 9, a description of the structure of the laminated body of the multilayered film optical filter 148 will be continued. In the above-described laminated body of the multilayered film optical filter 148 which is constituted by a multi-layered thin film, a first layer on the protection layer 136 side can be constituted by an oxide film such as silicon oxide (SiO₂). In this manner, the first layer on the protection layer 136 side, that is, the lowermost layer of the laminated body is constituted by an oxide film such as silicon oxide (SiO₂), and thus the layer has high adhesiveness with respect to a substrate (for example, the semiconductor substrate 141), which is generally used as abase material of the light receiving unit 140, or SiO₂ constituting the protection layer 136, which allows risk such as peeling to be suppressed.

Meanwhile, in the laminated body of the multilayered film optical filter 148 which is constituted by a multi-layered thin film, a first layer on the protection layer 136 side, that is, the lowermost layer of the laminated body can also be constituted by a nitride film such as silicon nitride (Si₃N₄). The nitride film has a higher refractive index than that of an oxide film. For this reason, the first layer as the lowermost layer which is located to be close to the photodiode element 135 is configured as a nitride film, and thus it is possible to more effectively reflect unnecessary light.

In addition, it is more preferable that the laminated body constituting the multilayered film optical filter 148 is formed of a multi-layered thin film of ten or more layers and 120 or less layers.

In this manner, the laminated body of the multilayered film optical filter 148 is constituted by ten or more layers, and thus it is possible to further efficiently cut unnecessary optical components (noise components). Specifically, it is possible to reduce the transmittance of light, having an unnecessary wavelength, which serves as a noise component to approximately 2%.

In addition, it is more preferable that the laminated body constituting the multilayered film optical filter 148 is formed of a multi-layered thin film of twenty or more layers and sixty or less layers.

In this manner, the laminated body of the multilayered film optical filter 148 is constituted by twenty or more layers, and thus it is possible to further efficiently cut unnecessary optical components (noise components). Specifically, it is possible to reduce the transmittance of light, having an unnecessary wavelength, which serves as a noise component to approximately 0.1%. In addition, the laminated body of the multilayered film optical filter 148 is constituted by sixty or less layers, and thus it is possible to further improve the productivity of the multilayered film optical filter 148.

In addition, it is more preferable that the laminated body constituting the multilayered film optical filter 148 is formed of a multi-layered thin film of twenty three or more layers and sixty or less layers.

In this manner, the laminated body of the multilayered film optical filter 148 is constituted by twenty three or more layers, and thus it is possible to further efficiently cut unnecessary optical components (noise components). Specifically, it is possible to reduce the transmittance of light, having an unnecessary wavelength, which serves as a noise component to approximately 0.05%. Therefore, it is possible to drastically reduce the transmittance of unnecessary optical components (noise components) while improving the productivity of the multilayered film optical filter with sixty or less layers laminated.

In addition, it is more preferable that the laminated body constituting the multilayered film optical filter 148 is formed of a multi-layered thin film of forty or more layers and sixty or less layers.

In this manner, the laminated body of the multilayered film optical filter 148 is constituted by forty or more layers, and thus it is possible to further efficiently cut unnecessary optical components (noise components). Specifically, it is possible to rigorously cut light, having a wavelength of 300 nm to 500 nm and having a wavelength of 600 nm to 1000 nm, which serves as a noise component during the measurement of pulse waves. Therefore, it is possible to drastically reduce light having a wavelength of 300 nm to 500 nm and having a wavelength of 600 nm to 1000 nm, which serves as a noise component during the measurement of pulse waves while improving the productivity of the multilayered film optical filter 148 with sixty or less layers laminated.

More preferably, the laminated body constituting the multilayered film optical filter 148 is formed of a multi-layered thin film of twenty three or more layers and forty or less layers.

In this manner, the laminated body of the multilayered film optical filter 148 is configured, and thus it is possible to reduce the transmittance of light, having an unnecessary wavelength, which serves as a noise component to approximately 0.05%, to efficiently cut light, having an unnecessary wavelength, which serves as a noise component, and to further improve the productivity of the multilayered film optical filter, thereby allowing production to be efficiently performed.

When a pulsimeter is taken as an example of the biological information measuring apparatus, light emitted from the light emitting unit 150 travels within a test subject which is an object, and is diffused or scattered to epidermis, dermis, subcutaneous tissue, and the like. Thereafter, the light reaches a blood vessel (part to be detected) and is reflected. At this time, a portion of the light is absorbed into the blood vessel. Since the absorption of the light at the blood vessel varies by the influence of pulses and the amount of reflected light also varies, the light receiving unit 140 receives the reflected light and detects variations in the amount of light, and thus it is possible to detect a pulse rate which is biological information, and the like.

Such a biological information measuring apparatus optically measures the blood flow under a skin surface and converts the blood flow into a signal to thereby obtain biological information such as pulse waves and pulses. Therefore, in order to improve the accuracy of measurement and portability, it is important to reduce a noise component such as disturbance light in a light path between the light emitting unit 150 and the light receiving unit 140 and to reduce light (direct light or the like) which is directly incident on the light receiving unit 140 from the light emitting unit 150. From such a viewpoint, the inventors have found that it is effective to provide a frame (wall portion 70) as a light shielding unit to be described below.

The wall portion 70 as a frame is mounted on a supporting surface 160a of the substrate 160 between the light receiving unit 140 and the light emitting unit 150. The wall portion 70 is provided in the form of a plate wall so as to extend in a Y-axis direction along the outer circumferential sides of the light receiving unit 140 and the light emitting unit 150 facing each other. The wall portion 70 abuts on a test subject which is an object, for example, the skin of the test subject on the top face (upper surface) thereof, and a desired space is formed on the upper surfaces of the light receiving unit 140 and the light emitting unit 150. In addition, the wall portion 70 shields, for example, light such as direct light which is directly incident on the light receiving unit 140 from the light emitting unit 150 or light such as disturbance light, serving as a noise component, which is incident on the light receiving unit 140. In this manner, the wall portion 70 is provided, and thus it is possible to prevent light emitted from the light emitting unit 150 from directly reaching (being incident on) the light receiving unit 140. Thereby, it is possible to allow light having less noise components to be incident on the light receiving unit 140 and to further improve the measurement accuracy of the biological information measuring module.

The wall portion 70 can be formed by, for example, sheet metal working of a metal plate. In this manner, when the wall portion 70 is formed by sheet metalworking of a metal plate, the wall portion 70 having excellent strength can be easily formed of an inexpensive material, and light can be reflected by the metallic wall portion 70, and thus it is possible to allow a test subject which is an object to be efficiently irradiated with light emitted from the light emitting unit 150 or to allow reflected light from the test subject to be efficiently incident on the light receiving unit 140. Meanwhile, an example of a material of the wall portion 70 includes a resin such as rubber (including a natural resin and a synthetic resin) as a material other than a metal material. These materials can be easily obtained at a low cost, and allows the wall portion 70 to be easily formed.

Meanwhile, in this Configuration Example 1, a description has been given of a configuration in which the wall portion 70 as a frame is provided between the light receiving unit 140 and the light emitting unit 150 and has a wall shape extending in a Y-axis direction, but the invention is not limited thereto. For example, as in a second embodiment to be described later, it is also possible to configure a ring-shaped frame (wall portion) which surrounds the outer circumference of the light receiving unit 140 or the light emitting unit 150. Even in such a configuration, the same effects as those described above are exhibited.

Connection terminals 274 electrically connected to a control unit, not shown in the drawing, are provided on the supporting surface 160a of the substrate 160 (sensor substrate) as a supporting unit. The connection terminal 274 is a terminal for electrical connection, and can be formed by applying gold (Au) plating to a metal layer, for example, a copper (Cu) layer. The connection terminal 274 is electrically connected to a connection terminal (not shown) provided on a rear surface 160b through a through-hole electrode (not shown) or the like. The connection terminals 274 and the connection terminal, not shown in the drawing, on the rear surface 160b side are provided on the substrate 160, and thus it is possible to compactly connect a supporting unit (substrate 160) and, for example, a control unit not shown in the drawing, or the like.

According to the configurations of the biological information measuring apparatus of the first embodiment described above and the sensor unit 40 as a biological information measuring module, the multilayered film optical filter 148 provided in the light receiving unit 140 is constituted by a laminated body having a predetermined number or more of layers laminated, and thus it is possible to cut optical components (noise components) which are unnecessary for the measurement of biological information such as pulse waves and to accurately measure biological information. In addition, the multilayered film optical filter is constituted by a laminated body having a predetermined number or less of layers laminated, it is possible to prevent a deterioration in productivity during the manufacture of the multilayered film optical filter 148. In this manner, it is possible to provide the biological information measuring apparatus capable of efficiently cutting optical components (noise components) which are unnecessary for the measurement of biological information and accurately measuring biological information, and the sensor unit 40 as a biological information measuring module.

Meanwhile, in the above-described configuration, a description has been given of an example in which the multilayered film optical filter 148 is formed of a laminated body (multi-layered thin film) which is laminated on the upper side of the semiconductor substrate 141 through the protection layer 136, but the invention is not limited to such a configuration. For example, it is also possible to apply a configuration in which the multilayered film optical filter 148 is formed of a laminated body (multi-layered thin film) in which a thin film is laminated on the upper side of the semiconductor substrate 141 through the angle limiting filter 142, which is a configuration in which the protection layer 136 is not provided.

### Example 1

As described above, the above-mentioned light receiving unit 140 in the sensor unit 40 can be configured as a light detecting apparatus capable of contributing to the realization of highly-accurate measurement of biological information in a biological information measuring apparatus. Hereinafter, an example of a light detecting apparatus will be described. Meanwhile, the example to be described below does not wrongly limit the contents of the invention described in the appended claims. In addition, the entire configuration described in this example is not necessarily an essential element of the invention.

The light detecting apparatus has a problem that the securing of moisture resistance and the securing of light transmittance are contrary to each other. For example, when moisture resistance is secured by simply protecting the light detecting apparatus by a silicon nitride film, light transmittance deteriorates.

Consequently, in this example, the entirety of a semiconductor substrate is covered with a multilayered film optical filter. In other words, the multilayered film optical filter is provided not only with a function of controlling light transmittance but also with a moisture-resisting function. As disclosed in the above-described invention of JP-A-6-21470, alternate layers of titanium oxide (TiO₂) and silicon oxide (SiO₂) have high moisture resistance. Accordingly, it is possible to achieve the maintenance of both moisture resistance and light transmittance without needing to add a new layer.

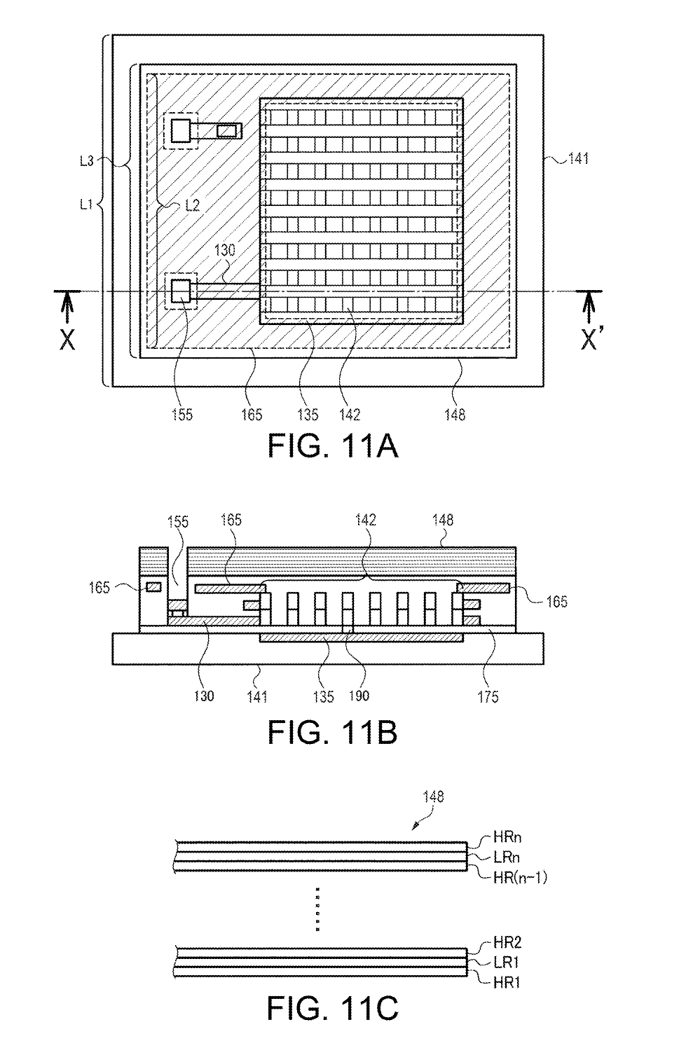

Specifically, a configuration of the light detecting apparatus of Example 1 is illustrated in FIGS. 11A to 11C. FIG. 11A is a schematic plan view of the light detecting apparatus, and FIG. 11B is a cross-sectional view taken along line X-X′ of FIG. 11A.

As illustrated in FIG. 11A, the light detecting apparatus of Example 1 includes a semiconductor substrate 141, a photodiode element 135 as a light detecting element formed on the surface of the semiconductor substrate 141, a wiring 130 formed on the semiconductor substrate 141, and a multilayered film optical filter 148 formed on the photodiode element 135 and the wiring 130. Further, the light detecting apparatus illustrated in FIGS. 11A and 11B may include a bonding pad 155 (hereinafter, referred to as a pad 155) to be described later, a light shielding layer (light shielding film) 165, an insulating layer 175 (silicon oxide film), an angle limiting filter 142, and a conductive plug 190. Meanwhile, the light detecting apparatus is not limited to the configurations illustrated in FIGS. 11A and 11B, and various modifications such as the omission of some of the components (angle limiting filter and the like) thereof, replacement with other components, or the addition of other components can be made.

The photodiode element 135 is constituted by an impurity region (for example, a diffusion layer) which is formed on the semiconductor substrate 141 (for example, a silicon substrate). When the impurity region is an N-type impurity region and the silicon substrate is a P type, a PN junction portion corresponds to the photodiode element 135.

Originally, the multilayered film optical filter 148 is a filter provided in order to limit a frequency band of light incident on the photodiode element 135. For example, not only reflected light of light emitted to an object from a light source but also external light or the like is incident on the light detecting apparatus, but such external light becomes noise when an electronic apparatus including the light detecting apparatus performs a measurement process. Consequently, the multilayered film optical filter 148 is formed on the upper surface of the photodiode element 135, and incident light is incident on the photodiode element 135 through the multilayered film optical filter 148. Thereby, it is possible to perform control so that only reflected light components of light emitted from a light source is incident on the photodiode element 135 by cutting external light components from incident light. Further, in a case where the light detecting apparatus is used for the purpose of measuring the inside of a human body, or the like, it is possible to limit a frequency band of incident light so that, for example, only light with a wavelength having a tendency to be absorbed into blood is transmitted.

In this example, the multilayered film optical filter 148 which is originally formed for the purpose of controlling light transmission is also provided with a role as a film improving moisture resistance.

Specifically, the multilayered film optical filter 148 is formed in such a way that the wiring 130 is positioned inside a region in which the multilayered film optical filter 148 is formed, when seen in a plan view in a direction perpendicular to the semiconductor substrate 141. In other words, the multilayered film optical filter 148 is provided so as to cover the wiring 130 and the photodiode element 135 when seen in a plan view in a direction perpendicular to the semiconductor substrate 141. Meanwhile, the wording “direction perpendicular to the semiconductor substrate 141” as used herein refers to a direction perpendicular to a surface on which a circuit is formed. In addition, the wording “a plan view in a direction perpendicular to the semiconductor substrate 141” as used herein refers to a plan view when the semiconductor substrate 141 is seen from a predetermined point positioned in a direction perpendicular to the semiconductor substrate 141.

As illustrated in an enlarged view of FIG. 11C, the multilayered film optical filter 148 is formed in such a way that a high refractive index layer (TiO₂) HRi and a low refractive index layer (SiO₂) LRi are alternately laminated on each other (i satisfies the relation of 1≦i≦n, and n is an integer of 2 or greater).

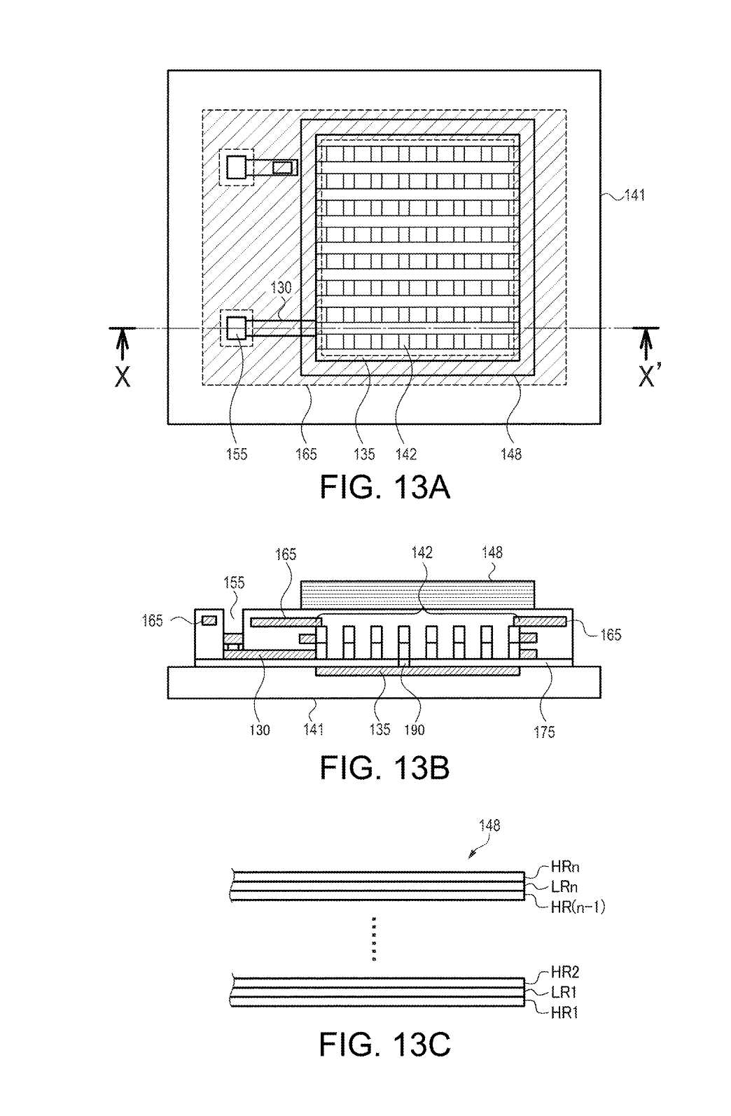

Thereby, it is possible to prevent the wiring 130 and the photodiode element 135 from being eroded by moisture. In addition, as described above, the multilayered film optical filter 148 can also maintain light transmittance. Further, in such a configuration, a new additional layer is not necessary, and thus a manufacturing cost does not increases compared to a configuration illustrated in FIG. 13A to be described later. Accordingly, it is possible to improve moisture resistance and light transmittance while suppressing a manufacturing cost. Meanwhile, the multilayered film optical filter 148 can be formed by a separate manufacturing process after the other portions of the light detecting apparatus are formed by a series of manufacturing processes. Meanwhile, the multilayered film optical filter 148 may be configured such that silicon nitride (Si₃N₄) HRi as a high refractive index layer and a low refractive index layer (SiO₂) LRi are alternately laminated on each other (i satisfies the relation of 1≦i≦n, and n is an integer of 2 or greater).

In addition, the light detecting apparatus of this example includes a pad 155 for external connection of the light detecting apparatus. The pad 155 has a structure for connecting an end of the photodiode element 135 to the outside (or a bonding wire).

Specifically, first, an end of the photodiode element 135 is electrically connected to the pad. The wording “an end of the photodiode element 135” as used herein refers to one terminal out of two terminals of a photodiode. More specifically, an end of the photodiode element 135 is an electrode (for example, a conductive plug 190) which is connected to an impurity region in which a photodiode is formed.

At this time, for example, the angle limiting filter 142 may be formed of the conductive plug 190. In this case, as illustrated in FIGS. 11A and 11B, the photodiode element 135 and the conductive plug 190 are electrically connected to each other, and the conductive plug 190 and the angle limiting filter 142 are electrically connected to each other. The angle limiting filter 142 and the wiring 130 which are disposed in the form of a lattice are electrically connected to each other, and thus an end of the photodiode element 135 and the pad are electrically connected to each other.

Alternatively, an end of the photodiode element 135 may be an electrode that applies a potential to the silicon substrate. Meanwhile, another circuit element (resistor or the like) may be interposed between the photodiode element 135 and the pad 155.

A bonding wire, not shown in the drawing, is electrically connected to the pad 155, and the bonding wire is electrically connected to a terminal, not shown in the drawing, of a chip of the light detecting apparatus. Further, the terminal is electrically connected to a wiring of an external circuit board not shown in the drawing.

Thereby, it is possible to electrically connect an end of the photodiode element 135 to the outside.

The multilayered film optical filter 148 is formed so as to have an opening at the position of the pad 155, when seen in a plan view in a direction perpendicular to the semiconductor substrate 141. In other words, the multilayered film optical filter 148 of this example does not necessarily have to be formed on the entire surface of the semiconductor substrate 141, and may have an opening or the like in a portion thereof.

Thereby, it is possible to prevent the multilayered film optical filter 148 from closing the pad 155. Therefore, as described above, it is possible to electrically connect a bonding wire to the pad 155.

In addition, the wiring 130 is a wiring for electrically connecting the pad 155 and an end of the photodiode element 135. Specifically, the wiring 130 is constituted by a metal layer such as aluminum on an insulating layer 175 (silicon oxide film).

Thereby, it is possible to electrically connect an external device and the photodiode element 135.

In addition, the light detecting apparatus of this example includes a light shielding layer 165 which is formed on the semiconductor substrate 141. The light shielding layer 165 is constituted by, for example, a metal layer. The multilayered film optical filter 148 is formed in such a way that the light shielding layer 165 is positioned inside a region in which the multilayered film optical filter 148 is formed, when seen in a plan view in a direction perpendicular to the semiconductor substrate 141. Further, the light shielding layer 165 is formed so as to be positioned outside a region in which the photodiode element 135 is formed. Further, the light shielding layer 165 is disposed between the semiconductor substrate 141 or the insulating layer 175 and the multilayered film optical filter 148 in a cross-sectional view.

Thereby, it is possible to allow incident light to be transmitted in a region in which the photodiode element 135 is formed and to be shielded in a region in which the photodiode element 135 is not formed. In other words, when seen in a plan view of the light detecting apparatus in the example of FIG. 11A, it is possible to allow light to be shielded only in a hatched region surrounded by a dotted line. For this reason, it is possible to prevent unnecessary charge from being generated in the semiconductor substrate 141 in the region in which the photodiode element 135 is not formed, and to prevent light from being incident on the photodiode element 135 from an angle falling outside a predetermined range.

In addition, the multilayered film optical filter 148 is provided in such a way that one side of the multilayered film optical filter 148 is positioned between one side of the light detecting apparatus and one side of the light shielding layer 165 which faces one side of the light detecting apparatus, when seen in a plan view in a direction perpendicular to the semiconductor substrate 141. For example, in the example of FIG. 11A, one side of the light detecting apparatus is L1, and one side of the light shielding layer 165 which faces the one side L1 of the light detecting apparatus is L2. One side of the multilayered film optical filter 148 which is positioned between the side L1 and the side L2 is L3.

Thereby, the multilayered film optical filter 148 is formed so as to cover the light shielding layer 165, and thus it is possible to prevent the occurrence of erosion due to moisture when the light shielding layer 165 is a metal layer.

In addition, the angle limiting filter 142 is formed of a light shielding material (light absorbing material or a light reflecting material) which has a light shielding property with respect to a wavelength detected by the photodiode element 135. Specifically, the angle limiting filter 142 is formed by a wiring forming step of a semiconductor process, and is constituted by, for example, a conductive layer such as an aluminum (light reflecting material) wiring layer and a conductive plug such as a tungsten (light absorbing material) plug.

### Modification Example

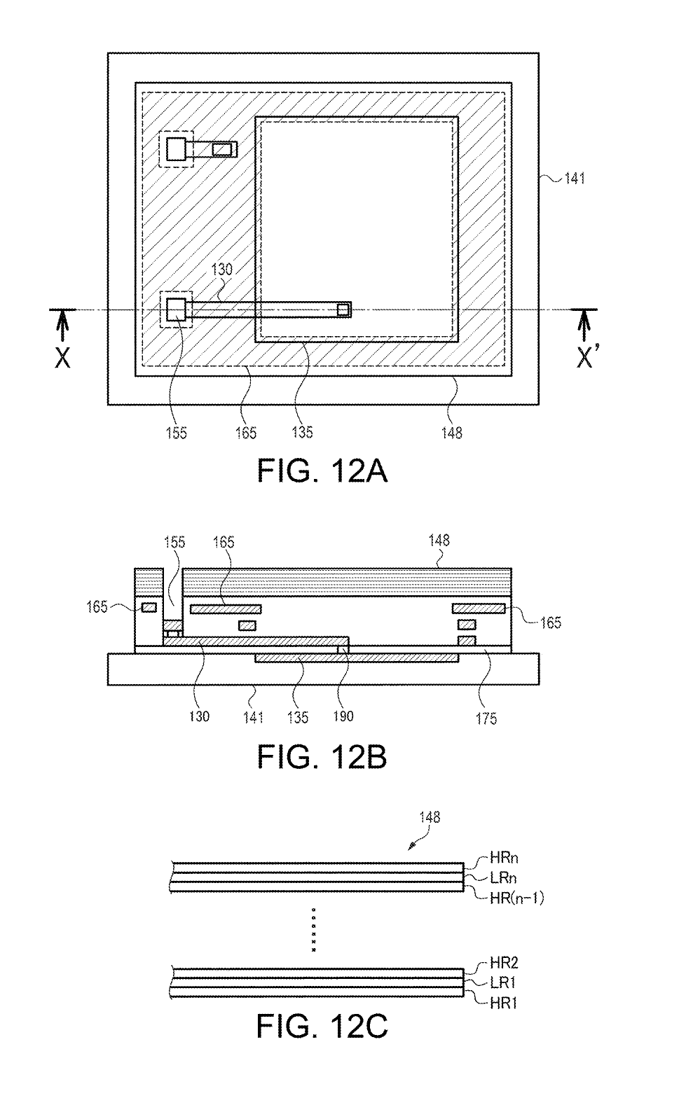

Here, the angle limiting filter 142 may not be necessarily provided in this example. For example, as illustrated in FIGS. 12A to 12C, the light detecting apparatus of this example may be configured such that the angle limiting filter 142 is not provided.

### Comparative Example

FIGS. 13A to 13C illustrate a light detecting apparatus as a comparative example of this example. The light detecting apparatus of the comparative example illustrated in FIG. 13A includes a semiconductor substrate 141, a photodiode element 135, a wiring 130, a multilayered film optical filter 148, a pad 155 for bonding, a light shielding layer 165, an insulating layer 175 (silicon oxide film), an angle limiting filter 142, and a conductive plug 190, as illustrated in FIGS. 13A and 13B.

As illustrated in FIG. 13C, a configuration of the multilayered film optical filter 148 is the same as the configuration illustrated in FIG. 11C. Here, unlike FIG. 11C, in the light detecting apparatus illustrated in FIGS. 13A to 13C, the multilayered film optical filter 148 does not cover the entire semiconductor substrate. Specifically, in the example of FIG. 13A, the multilayered film optical filter 148 covers only the photodiode element 135 and the angle limiting filter 142, and does not cover the wiring 130, the light shielding layer 165, and the like. For this reason, there is a concern of the wiring 130, the light shielding layer 165, and the like being eroded by moisture, and thus there is a problem in moisture resistance. On the other hand, as described above, the light detecting apparatus illustrated in FIG. 11A is configured such that the multilayered film optical filter 148 is formed so as to cover the wiring 130, thereby solving the problem of moisture resistance.

### Example 2

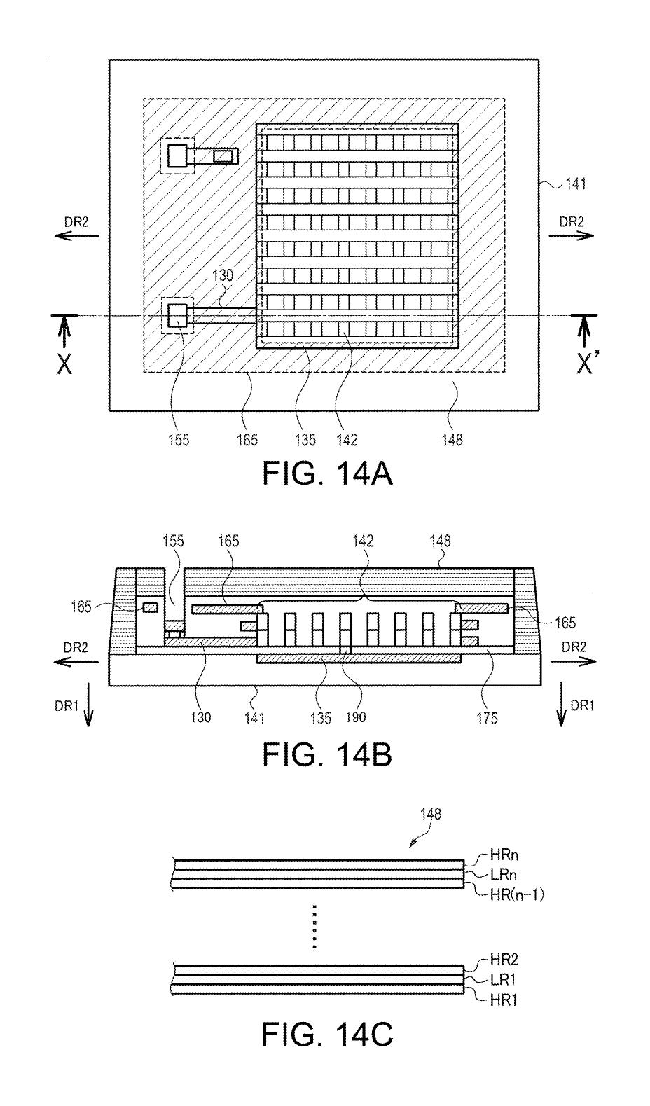

Next, FIGS. 14A to 14C illustrate Example 2. In Example 1 described above, a multilayered film optical filter 148 is provided so as to cover at least a photodiode element 135 and a wiring 130 on a semiconductor substrate 141. In other words, the multilayered film optical filter 148 is formed only on the opposite upper surface of the light detecting apparatus when the light detecting apparatus is seen in a plan view.

On the other hand, in Example 2, as illustrated in FIGS. 14A and 14B, the multilayered film optical filter 148 is formed not only on the upper surface of the light detecting apparatus but also on the side surface thereof. In other words, when a direction when seen in a plan view in a direction perpendicular to the semiconductor substrate 141 is set to be a first direction DR1 and a direction perpendicular to the first direction DR1 is set to be a second direction DR2, the multilayered film optical filter 148 may be formed on the second direction DR2 side of the insulating layer 175 at an end of an insulating layer 175.

Thereby, it is possible to prevent water from entering the inside of the light detecting apparatus from the side surface of the apparatus. Meanwhile, as illustrated in FIG. 14C, the multilayered film optical filter 148 at this time has the same laminated structure as that in FIG. 11C.

### Configuration Examples of Light Detecting Module and Electronic Apparatus

The light detecting apparatus of the above-mentioned example is included in a light detecting module or an electronic apparatus. For example, the light detecting module includes a light detecting apparatus and a light emitting unit. In addition, the electronic apparatus is, for example, a measuring apparatus or a biological information detecting apparatus.

For example, when the electronic apparatus is a measuring apparatus, the measuring apparatus (biological information detecting apparatus) of FIGS. 1 to 3 can be adopted as an embodiment. In this measuring apparatus, the sensor unit 40 as a biological information measuring module which is illustrated in FIG. 3 functions as a light detecting module. The light detecting module (sensor unit 40) is a module that detects measured biological information such as pulse waves of a test subject.

### Another Configuration Example of Sensor Unit

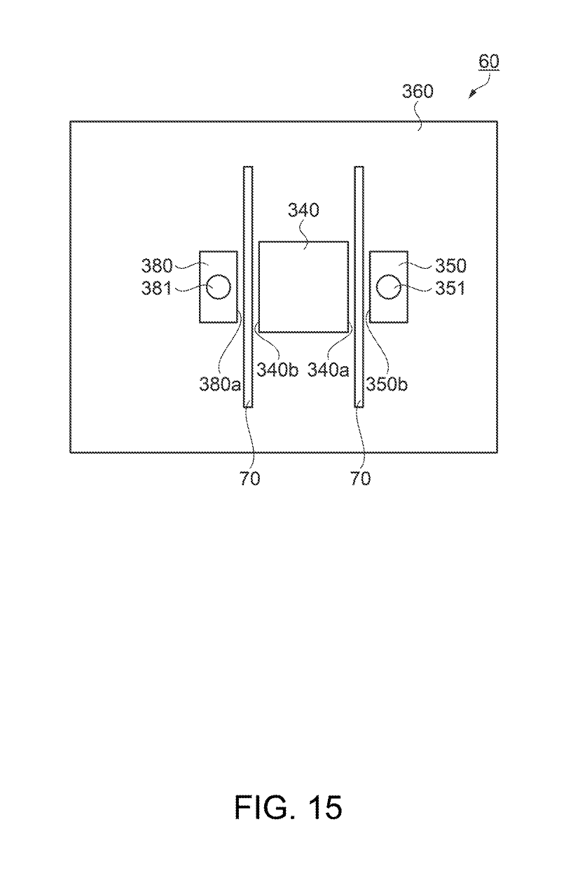

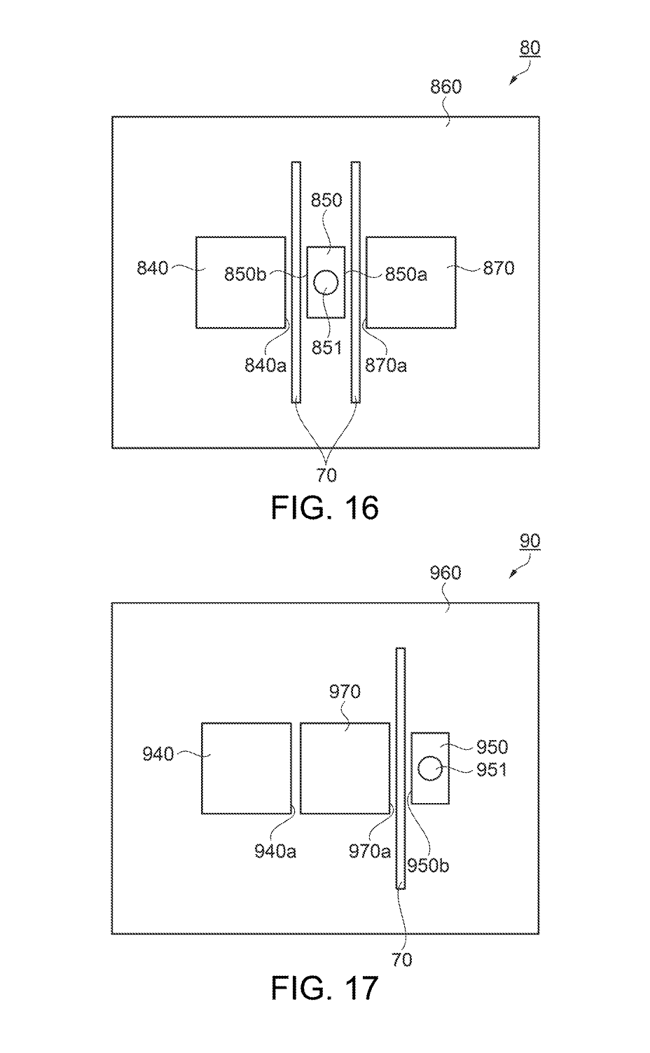

Next, another configuration example of the sensor unit 40 described above will be described with reference to FIGS. 15 to 17. FIG. 15 is a plan view illustrating Configuration Example 2 of the sensor unit. In addition, FIG. 16 is a plan view illustrating Configuration Example 3, and FIG. 17 is a plan view illustrating Configuration Example 4. Meanwhile, in FIGS. 15 to 17, the arrangement of a light receiving unit 140, a light emitting unit 150, and a wall portion 70 as a frame is mainly illustrated, and the other components are not illustrated. In addition, the same components as those in the first embodiment described above are denoted by the same reference numerals and signs, and a description thereof may be omitted. Also in the following configuration examples, for example, the structure of the multilayered film optical filter 148 which is a component of the light receiving unit 140 described above can be similarly applied.

### Configuration Example 2

First, a sensor unit 60 according to Configuration Example 2 will be described with reference to FIG. 15. In Configuration Example 1 of the first embodiment described above, one light emitting unit 150 and one light receiving unit 140 are mounted on a substrate 160 (sensor substrate) so as to be lined up. In a configuration of the sensor unit 60 according to this Configuration Example 2, a plurality of light emitting units (a first light emitting unit 350 and a second light emitting unit 380) and one light receiving unit 340 are provided such that the first light emitting unit 350 and the second light emitting unit 380 as a plurality of light emitting units and the light receiving unit 340 are mounted on a substrate 360 so as to be lined up in a row along a predetermined direction in the order of the first light emitting unit 350, the light receiving unit 340, and the second light emitting unit 380. A wall portion 70 as a frame is provided between the first light emitting unit 350 and the light receiving unit 340 and between the second light emitting unit 380 and the light receiving unit 340.

Specifically, it is preferable that the first light emitting unit 350, the light receiving unit 340, and the second light emitting unit 380 are disposed so that a distance between an outer circumferential side 350b of the first light emitting unit 350 on the light receiving unit 340 side and an outer circumferential side 340a of the light receiving unit 340 on the first light emitting unit 350 side is set to be equal to a distance between an outer circumferential side 380a of the second light emitting unit 380 on the light receiving unit 340 side and an outer circumferential side 340b of the light receiving unit 340 on the second light emitting unit 380 side.

According to the sensor unit 60 of Configuration Example 2, a plurality of light emitting units (in this example, the first light emitting unit 350 and the second light emitting unit 380) are provided, and thus it is possible to secure sufficient light emission intensity by light emitted from the plurality of light emitting units. In addition, biological information is detected by detecting light from the plurality of light emitting units, and thus it is possible to further improve measurement accuracy.

By this arrangement, the length of a light path between the first light emitting unit 350 and the light receiving unit 340 and the length of a light path between the second light emitting unit 380 and the light receiving unit 340 are set to be substantially the same as each other, and light beams emitted from the first light emitting unit 350 and the second light emitting unit 380 are incident on the light receiving unit 340 at substantially the same time, and thus it is possible to improve an S/N ratio. That is, it is possible to improve the measurement accuracy of the biological information measuring apparatus.

Meanwhile, in Configuration Example 2 described above, a description has been given of a configuration in which the wall portion 70 as a frame has a wall shape extending in one direction, but the invention is not limited thereto. For example, as in a second embodiment to be described later, it is also possible to configure a ring-shaped frame (wall portion) which surrounds the outer circumference of the light receiving unit 340 or the light emitting unit 350. Even in such a configuration, the same effects as those described above are exhibited.

### Configuration Example 3

Next, a sensor unit 80 according to Configuration Example 3 will be described with reference to FIG. 16. In a configuration of the sensor unit 80 according to Configuration Example 3, after a first light receiving unit 840 and a second light receiving unit 870 as light receiving units share alight emitting unit 850, one first light receiving unit 840 and one second light receiving unit 870 are respectively disposed on both sides of the light emitting unit 850 along a predetermined direction and are mounted on a substrate 860 so as to be lined up in a row. A wall portion 70 as a frame is provided between the light emitting unit 850 and the first light receiving unit 840 and between the light emitting unit 850 and the second light receiving unit 870. Meanwhile, the units are disposed so that an interval between the light emitting unit 850 and the first light receiving unit 840 and an interval between the light emitting unit 850 and the second light receiving unit 870 are set to be substantially the same distance. Specifically, a distance between an outer circumferential side 850b of the light emitting unit 850 on the first light receiving unit 840 side and an outer circumferential side 840a of the first light receiving unit 840 on the light emitting unit 850 side is substantially the same as a distance between an outer circumferential side 850a of the light emitting unit 850 on the second light receiving unit 870 side and an outer circumferential side 870a of the second light receiving unit 870 on the light emitting unit 850 side.

According to the sensor unit 80 of Configuration Example 3, a plurality of light receiving units (in this example, the first light receiving unit 840 and the second light receiving unit 870) are provided, and thus it is possible to receive a greater amount of light (light with high intensity) and to improve measurement accuracy.

In addition, similarly to Configuration Example 2 described above, the length of a light path between the light emitting unit 850 and the first light receiving unit 840 and the length of a light path between the light emitting unit 850 and the second light receiving unit 870 are set to be substantially the same as each other, and light beams emitted from the light emitting unit 850 are incident on the first light receiving unit 840 and the second light receiving unit 870 at substantially the same time, and thus it is possible to improve an S/N ratio. That is, it is possible to improve the measurement accuracy of the biological information measuring apparatus.

### Configuration Example 4

Next, a sensor unit 90 according to Configuration Example 4 will be described with reference to FIG. 17. In a configuration of the sensor unit 90 according to Configuration Example 4, after a first light receiving unit 940 and a second light receiving unit 970 as light receiving units share a light emitting unit 950, the units are mounted on a substrate 960 so as to be lined up in a row along a predetermined direction in the order of the light emitting unit 950, the second light receiving unit 970, and the first light receiving unit 940. Therefore, the light emitting unit 950, the second light receiving unit 970, and the first light receiving unit 940 are disposed so that an interval between the light emitting unit 950 and the first light receiving unit 940 and an interval between the light emitting unit 950 and the second light receiving unit 970 are set to be substantially the same distance. A wall portion 70 as a frame is provided between the light emitting unit 950 and the second light receiving unit 940.

Specifically, a distance between an outer circumferential side 950b of the light emitting unit 950 on the first light receiving unit 940 side and an outer circumferential side 940a of the first light receiving unit 940 on the light emitting unit 950 side is different from a distance between an outer circumferential side 950b of the light emitting unit 950 on the second light receiving unit 970 side and an outer circumferential side 970a of the second light receiving unit 970 on the light emitting unit 950 side. In other words, a distance between the first light receiving unit 940 and the light emitting unit 950 is longer than a distance between the second light receiving unit 970 and the light emitting unit 950.

According to the sensor unit 90 of Configuration Example 4, the length of a light path between the light emitting unit 950 and the first light receiving unit 940 and the length of a light path between the light emitting unit 950 and the second light receiving unit 970 are different from each other, and a timing at which light emitted from the light emitting unit 950 is incident on the first light receiving unit 940 and a timing at which light emitted from the light emitting unit is incident on the second light receiving unit 970 are different from each other, and thus it is possible to acquire a larger amount of biological information.

Meanwhile, in Configuration Examples 3 and 4 mentioned above, a description has been given of a configuration in which the wall portion 70 as a frame has a wall shape extending in one direction, but the invention is not limited thereto. For example, as in the second embodiment to be described later, it is also possible to configure a ring-shaped frame (wall portion) which surrounds the outer circumference of each of the first light receiving units 840 and 940, the second light receiving units 870 and 970, or the light emitting units 850 and 950. Even in such a configuration, the same effects as those described above are exhibited.

### Second Embodiment

Next, the second embodiment of the invention will be described with reference to the accompanying drawings.

Similarly to the first embodiment described above, the biological information measuring apparatus according to the second embodiment is a heart rate monitoring apparatus which is worn on a living body (for example, a human body) of which biological information is measured, and which measures biological information such as a pulse (heart rate). Meanwhile, in the following drawings, each component has a size to the extent that the component can be recognized in the drawing, and thus a description may be given by appropriately making a dimension and proportion of each component different from those of an actual component. Also in the second to fifth embodiments, for example, the structure of the multilayered film optical filter 148 which is a component of the light receiving unit 140 described in the first embodiment can be similarly applied.

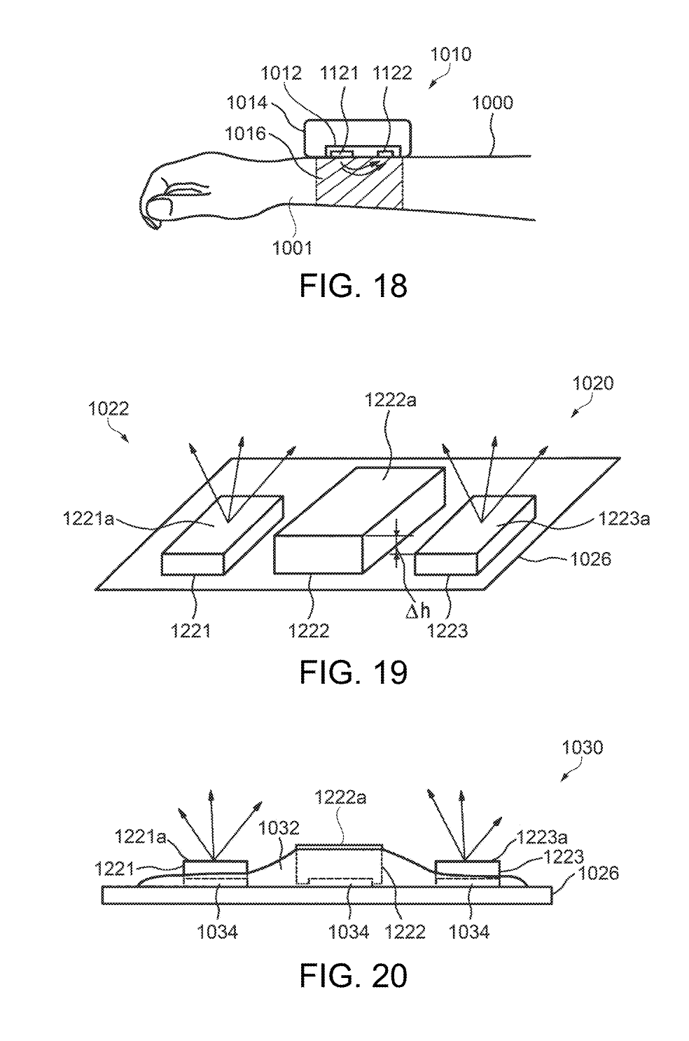

First, before a heart rate monitoring apparatus 1010 as the biological information measuring apparatus according to the second embodiment is described, an example of the art of the heart rate monitoring apparatus as the biological information measuring apparatus according to the second embodiment will be described with reference to FIG. 18.

FIG. 18 is a cross-sectional view illustrating a heart rate monitoring apparatus 1010 as a biological information measuring apparatus according to an example of the art which measures a physiologic parameter (biological information) of a user (test subject) 1000 (the user's arm is shown in the drawing) who is wearing the heart rate monitoring apparatus. The heart rate monitoring apparatus 1010 includes a sensor 1012 that measures a heart rate as at least one physiologic parameter of the user 1000, and a case 1014 that accommodates the sensor 1012. The heart rate monitoring apparatus 1010 is worn on the arm 1001 of the user 1000 by a fixation portion 1016 (for example, a band).

The sensor 1012 is a heart rate monitoring sensor that includes a light emitting element 1121 as a light emitting unit and a light receiving element 1122 as a light receiving unit which are two sensor elements and measures or monitors a heart rate. However, the sensor may be a sensor that measures one or more physiologic parameters (for example, a heart rate, blood pressure, the amount of air inhaled, skin conductivity, skin humidity, and the like). In addition, when the case 1014 includes a band-type housing, the heart rate monitoring apparatus can be used as a wristwatch type monitoring apparatus which is used in, for example, sport. Meanwhile, the case 1014 may have a shape capable of mainly holding the sensor 1012 at a desired position with respect to the user 1000, and may be able to arbitrarily accommodate more elements such as a battery, a processing unit, a display, and a user interface.

The biological information measuring apparatus of the existing example is the heart rate monitoring apparatus 1010 for monitoring a user's heart rate. The sensor 1012 is an optical sensor constituted by the light emitting element 1121 and the light receiving element 1122. An optical heart rate monitor using the optical sensor depends on the light emitting element 1121 (LED is generally used) as a light source that exposes the skin to light. The light emitted from the light emitting element 1121 to the skin is partially absorbed by blood flowing through a blood vessel under the skin, but the rest of the light is reflected and leaves the skin. The reflected light passes through a multilayered film optical filter provided on the light receiving element 1122 (photodiode is generally used) and is captured by the light receiving element 1122. A light reception signal from the light receiving element 1122 is a signal including information equivalent to the amount of blood flowing through the blood vessel. The amount of blood flowing through the blood vessel varies depending on pulse of the heart. In this manner, a signal on the light receiving element 1122 varies in response to the pulsation of the heart. In other words, a variation in the signal of the light receiving element 1122 is equivalent to the pulse of a heart rate. A pulse rate per unit time is counted (for example, per 10 seconds), to thereby obtain the number of beats of the heart for one minute (that is, a heart rate).

Hereinafter, a heart rate monitoring apparatus 1020 as the biological information measuring apparatus according to the second embodiment will be described with reference to FIG. 19. FIG. 19 is a perspective view illustrating a heart rate monitoring apparatus as the biological information measuring apparatus according to the second embodiment. Although not shown in FIG. 19, the heart rate monitoring apparatus 1020 as the biological information measuring apparatus according to the second embodiment is worn on a user's arm by a fixation portion such as a band, similar to the first embodiment described above.

In the heart rate monitoring apparatus 1020 as the biological information measuring apparatus according to the second embodiment, light emitting elements 1221 and 1223 as a plurality of (two in this example) light emitting units and a light receiving element 1222 as one light receiving unit are disposed so as to be lined up in a row. Specifically, a sensor 1022 (in this example, two light emitting elements 1221 and 1223 as a first light emitting unit and a second light emitting unit and the light receiving element 1222 as a light receiving unit are used as three sensor elements) which includes at least two sensor elements is provided. Meanwhile, although not shown in the drawing, it is preferable that a wall portion 70 (see FIG. 16) having the same configuration as that in Configuration Example 3 described above is provided between the light receiving element 1222 and the light emitting element 1221 and between the light receiving element 1222 and the light emitting element 1223.

The light receiving element 1222 as the light receiving unit is disposed between the two light emitting elements 1221 and 1223 as the first light emitting unit and the second light emitting unit. In addition, two light emitting elements 1221 and 1223 as the first light emitting unit and the second light emitting unit are disposed at line symmetrical positions with respect to a virtual line passing through the center of the light receiving element 1222 as the light receiving unit. The light emitting elements 1221 and 1223 and the light receiving element 1222 are disposed in such a manner, and thus it is possible to reduce dead space and to achieve space saving. In addition, light beams from both the first light emitting unit and the second light emitting unit, which are located at line symmetrical positions, gather in the light receiving unit, and thus detection can be performed more accurately.

The sensor element detects a sensor signal. The sensor 1022 includes an optical sensor constituted by the light emitting elements 1221 and 1223 using two LEDs for emitting light to the skin of a user, and a photodiode provided with at least one light receiving element 1222 (multilayered film optical filter) for receiving the light reflected from the skin. Further, the heart rate monitoring apparatus 1020 includes a case or a housing (not shown). The case or the housing may be similar to or the same as the case 1014 illustrated in FIG. 18, or may be similar to or the same as the case portion 30 in the first embodiment described above.

The sensor 1022 is carried on one surface of a carrier (substrate) 1026. Here, a configuration including the carrier (substrate) 1026 and the sensor 1022 carried on the carrier (substrate) 1026 corresponds to a biological information measuring module. Meanwhile, the same is true of the third to fifth embodiments. Light emitted from the light emitting elements 1221 and 1223 can be reflected without being absorbed into the skin or the like, and can directly reach the light receiving element 1222. In other words, light including a user's biological information directly reaches the multilayered film optical filter functioning as a wavelength limiting filter, passes through the multilayered film optical filter, and is incident on a photodiode. In the heart rate monitoring apparatus 1020, a distance between the carrier 1026 and each of upper surfaces 1221a and 1223a of the respective light emitting elements 1221 and 1223 is smaller than a distance between the carrier 1026 and an upper surface 1222a of the light receiving element 1222. That is, a difference between the distance between the carrier 1026 and each of the upper surfaces 1221a and 1223a of the respective light emitting elements 1221 and 1223 and the distance between the carrier 1026 and an upper surface 1222a of the light receiving element 1222 is Δh. The light receiving element 1222 receives light from the upper surface 1222a thereof which is the uppermost surface layer. According to these configurations, there is an effect that the most of light emitted from the light emitting elements 1221 and 1223 is directed to the skin and reflected light is directly incident on the light receiving element 1222 without going through an air layer or the like. In other words, since a structure in which the light receiving element 1222 comes into close contact with the skin is formed, a structure in which a gap is not likely to be generated between the upper surface (light receiving surface) 1222a of the light receiving element 1222 and the skin can be formed, and thus it is possible to prevent light, such as external light, which serves as a noise source from being incident on the upper surface 1222a. In addition, light from the light emitting elements 1221 and 1223 which does not pass through the skin, for example, light being directly incident on the light receiving element 1222 from the light emitting elements 1221 and 1223 cannot reach the upper surface 1222a of the light receiving element 1222.

### Third Embodiment

Next, a heart rate monitoring apparatus 1030 as the biological information measuring apparatus according to the third embodiment will be described with reference to FIG. 20. FIG. 20 is a front view illustrating a heart rate monitoring apparatus as the biological information measuring apparatus according to the third embodiment. Meanwhile, although not shown in FIG. 20, the heart rate monitoring apparatus 1030 as the biological information measuring apparatus according to the third embodiment is worn on a user's arm by a fixation portion such as a band, similar to the first embodiment described above.

As illustrated in FIG. 20, electric connection terminals 1034 of light emitting elements 1221 and 1223 as light emitting units and a light receiving element 1222 as a light receiving unit have to be preferably covered with an insulating material (for example, epoxy resin) 1032 in order to protect electrical elements. In addition, a configuration can be adopted in which the insulating material 1032 does not cover the light emitting elements 1221 and 1223 and the light receiving element 1222. Specifically, a configuration can be adopted in which the insulating material 1032 is buried in a region between the light emitting element 1221 and the light receiving element 1222 and a region between the light emitting element 1223 and the light receiving element 1222. In other words, a configuration can be adopted in which at least an upper surface 1222a of the light receiving element 1222 and upper surfaces 1221a and 1223a of the light emitting elements 1221 and 1223 are not covered with the insulating material 1032. With such a configuration, it is possible to suppress disturbance due to an air gap between the skin and the light emitting elements 1221 and 1223. Further, a configuration may be adopted in which the insulating material 1032 covers the upper surfaces 1221a and 1223a of the light emitting elements 1221 and 1223 and the upper surface 1222a of the light receiving element 1222. With such a configuration, the upper surface 1222a of the light receiving element 1222 which comes into contact with the skin and the upper surfaces 1221a and 1223a of the light emitting elements 1221 and 1223 can be protected, and thus it is possible to prevent the upper surface 1222a of the light receiving element 1222 and the upper surfaces 1221a and 1223a of the light emitting elements 1221 and 1223 from being damaged. In this case, the insulating material 1032 can be regarded as a protection film.

In the heart rate monitoring apparatus 1030 as the biological information measuring apparatus according to this third embodiment, the insulating material 1032 using an epoxy resin is provided, as an example which is generally implementable. In FIG. 20, the insulating material 1032 is disposed so as not to cover the upper surfaces 1221a and 1223a of the light emitting elements 1221 and 1223, and protects the electric connection terminals 1034. Light beams emitted from the light emitting elements 1221 and 1223 are indicated by an arrow.

In this manner, the insulating material 1032 is minimally disposed to the extent that a correct function of the heart rate monitoring apparatus 1030 is not hindered, and thus the heart rate monitoring apparatus 1030 can be further improved by protecting the electric connection terminals 1034 of the light emitting elements 1221 and 1223 and the light receiving element 1222. Meanwhile, although not shown in the drawing, it is more preferable that a wall portion 70 (see FIG. 16) having the same configuration as that in Configuration Example 3 described above is provided between the light receiving element 1222 and the light emitting element 1221 and between the light receiving element 1222 and the light emitting element 1223.

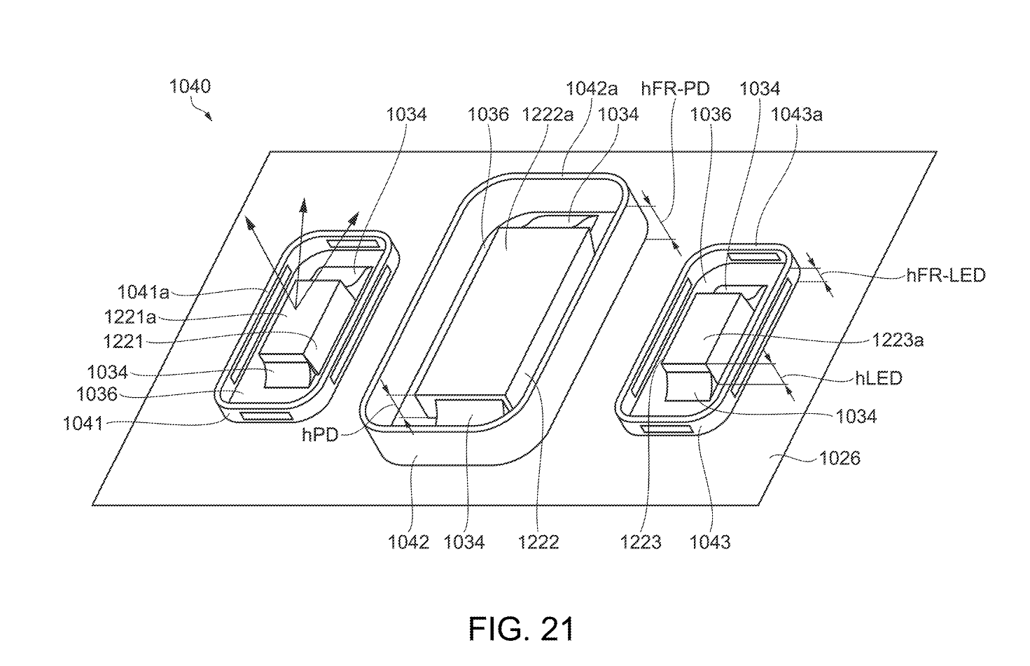

Meanwhile, it is more preferable to configure a heart rate monitoring apparatus 1040 as the biological information measuring apparatus according to the fourth embodiment as illustrated in FIG. 21, instead of adopting the configuration in this third embodiment in which an epoxy resin is injected.

### Fourth Embodiment

Next, a heart rate monitoring apparatus 1040 as the biological information measuring apparatus according to the fourth embodiment will be described with reference to FIG. 21. FIG. 21 is a perspective view illustrating a heart rate monitoring apparatus as the biological information measuring apparatus according to the fourth embodiment. Meanwhile, although not shown in FIG. 21, the heart rate monitoring apparatus 1040 as the biological information measuring apparatus according to the fourth embodiment is worn on a user's arm by a fixation portion, such as a band, similar to the first embodiment described above.

In the heart rate monitoring apparatus 1040 as the biological information measuring apparatus according to the fourth embodiment, frames 1041, 1042, and 1043 created are disposed. The frames 1041, 1042, and 1043 are disposed in the vicinity of the light emitting elements 1221 and 1223 as light emitting units and the light receiving element 1222 as a light receiving unit, and a space 1036 is formed between each of the frames 1041, 1042, and 1043 and each of the light emitting elements 1221 and 1223 and the light receiving element 1222. An insulating material (not shown in FIG. 21) is injected with the frames 1041, 1042, and 1043 as guides to cover the electric connection terminals 1034 of the light emitting elements 1221 and 1223 and the light receiving element 1222.

In the example shown in the fourth embodiment, the light emitting elements 1221 and 1223 and the light receiving element 1222 are surrounded by the respective frames 1041, 1042, and 1043. Meanwhile, as another example, all of the frames 1041, 1042, and 1043 may be coupled to each other, or all of the sensor elements may be surrounded by an integrated frame. Meanwhile, the frames 1041, 1042, and 1043 can be used as light shielding walls (wall portions) as examples of light shielding units. The frames 1041, 1042, and 1043 are used as light shielding walls (wall portions), and thus it is possible to prevent light emitted from the light emitting elements 1221 and 1223 from being directly incident on the light receiving element 1222.

As an improvement for preventing the function of the heart rate monitoring apparatus 1040 from being affected, it is preferable that upper edges 1041a and 1043a of the frames 1041 and 1043 in the vicinity of the light emitting elements 1221 and 1223 are lower than the upper surfaces 1221a and 1223a of the light emitting elements 1221 and 1223. In other words, a distance hFR−LED between the carrier 1026 and each of the upper edges 1041a and 1043a of the respective frames 1041 and 1043 is the same as or smaller than a distance hLED between the carrier 1026 and each of the upper surfaces 1221a and 1223a of the light emitting elements 1221 and 1223 which are surrounded by the respective frames 1041 and 1043 (hFR−LED≦hLED).

It is preferable that a difference between the distance hLED between the carrier 1026 and each of the upper surfaces 1221a and 1223a of the respective light emitting elements 1221 and 1223 and the distance hFR−LED between the carrier 1026 and each of the upper edges 1041a and 1043a of the respective frames 1041 and 1043 is set to be in a range from 0.1 mm to 0.8 mm. Meanwhile, it is more preferable that a difference between the distance hLED between the carrier 1026 of each of the upper surfaces 1221a and 1223a of the respective light emitting elements 1221 and 1223 and the distance hFR−LED between the carrier 1026 and each of the upper edges 1041a and 1043a of the respective frames 1041 and 1043 is set to be in a range from 0.2 mm to 0.5 mm.

In addition, it is preferable that an upper edge 1042a of the frame (receiver frame) 1042 in the vicinity of the light receiving element 1222 is higher than the upper surface 1222a of the light receiving element 1222. In other words, a distance hFR−PD between the carrier 1026 and the upper edge 1042a of the frame 1042 is larger than a distance hPD between the carrier 1026 and the upper surface 1222a of the light receiving element 1222 surrounded by the frame 1042 (hFR−PD>hPD).

It is preferable that a difference between the distance hPD between the carrier 1026 and the upper surface 1222a of the light receiving element 1222 and the distance hFR−PD between the carrier 1026 and the upper edge 1042a of the frame 1042 is set to be in a range from 0 mm to 0.5 mm. Meanwhile, it is more preferable that a difference between the distance hPD between the carrier 1026 and the upper surface 1222a of the light receiving element 1222 and the distance hFR−PD between the carrier 1026 and the upper edge 1042a of the frame 1042 is set to be in a range from 0.1 mm to 0.2 mm.

Further, the distance hFR−PD between the carrier 1026 and the upper edge 1042a of the frame 1042 is larger than the distance hLED between the carrier 1026 and the upper surfaces 1221a and 1223a of the respective light emitting elements 1221 and 1223 (hFR−PD>hLED).

Meanwhile, for example, when the light receiving element 1222 and the light emitting elements 1221 and 1223 are close to each other, a configuration may be adopted in which only one frame wall is present between the light receiving element 1222 and each of the light emitting elements 1221 and 1223. This may occur because of manufacturing easiness. When the one frame wall is a case, frame walls of the frames of both the light receiving element 1222 and each of the light emitting elements 1221 and 1223 are coincident with each other. This means that the frame walls of the light emitting elements 1221 and 1223 become relatively high. In detail, the frame wall on the light receiving element 1222 side of the frames 1041 and 1043 surrounding the respective light emitting elements 1221 and 1223 become relatively high, and the other frame wall becomes lower than the upper surfaces 1221a and 1223a of the respective light emitting elements 1221 and 1223.

Further, instead of the frames 1041, 1042, and 1043, a configuration may be adopted in which a first wall portion is provided between the light receiving element 1222 and the light emitting element 1221 or the light emitting element 1223 and a second wall portion is provided on the outside of the light emitting elements 1221 and 1223, that is, on the side opposite to the first wall portion with respect to the light receiving element 1222.

In such a configuration, a distance between the carrier 1026 and the upper surface of the first wall portion may be larger than a distance between the carrier 1026 and the upper surface of the second wall portion. With such a configuration, it is possible to realize the function of the frame using a smaller number of members than in a case where a light emitting element and a light receiving element are surrounded as illustrated in FIG. 21.

Meanwhile, the frames 1041 and 1043 and the frame 1042 are used as in this fourth embodiment, and thus it is possible to prevent an insulating material to be injected, such as an epoxy resin, from flowing out. In this manner, the partitioning of an insulating material such as an epoxy resin by creating an additional structure is option of allowing high mass productivity to be obtained. Meanwhile, the frames 1041 and 1043 and the frame 1042 may be formed of the same material as that of the carrier 1026. For example, the frames may be formed by injection molding using an epoxy-based resin or a polycarbonate-based resin.

As described above, the insulating material 1032 (see FIG. 20) protects the electric connection terminals 1034 of the sensor elements (light emitting elements 1221 and 1223 and the light receiving element 1222). However, the electric connection terminals 1034 have to further come into contact with additional electronic apparatuses (for example, a driver, detection electronics, a processor, or a power supply) which are other elements. This means that there is any electrical connection between the carrier 1026 (may be a printed circuit board (PCB)) and the additional electronic apparatuses. In addition, the structure of the heart rate monitoring apparatus according to this embodiment can be applied not only to an apparatus for measuring a heart rate but also to apparatuses for measuring pulse waves and pulse.

### Fifth Embodiment

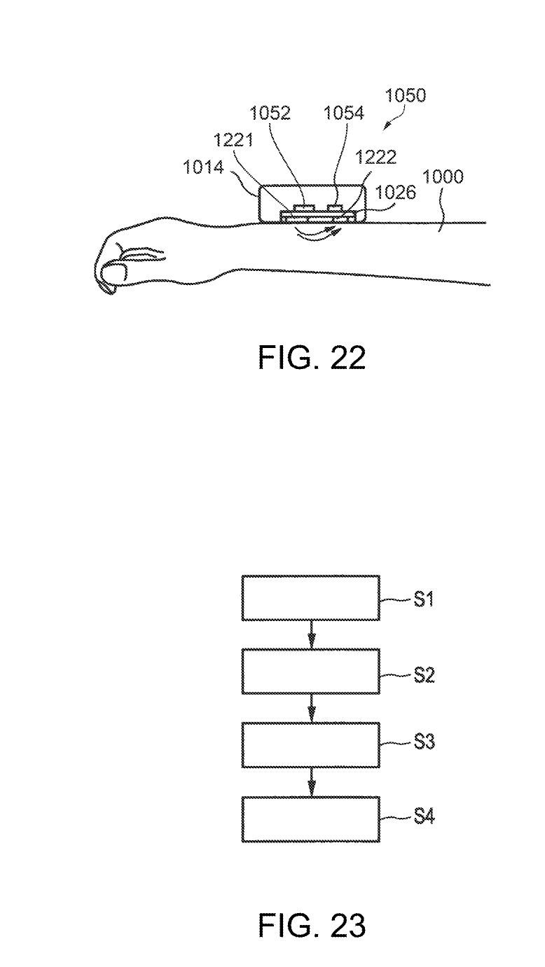

A heart rate monitoring apparatus 1050 as the biological information measuring apparatus according to the fifth embodiment will be described with reference to FIG. 22. FIG. 22 is a cross-sectional view illustrating a heart rate monitoring apparatus as the biological information measuring apparatus according to the fifth embodiment. Meanwhile, although not shown in FIG. 22, the heart rate monitoring apparatus 1050 as the biological information measuring apparatus according to the fifth embodiment is worn on a user's arm by a fixation portion such as a band, similar to the first embodiment described above.

The heart rate monitoring apparatus 1050 as the biological information measuring apparatus according to the fifth embodiment includes the above-mentioned additional electronic apparatuses (for example, a processor 1052 and a driver 1054). An external electric connection terminal (not shown) is not disposed on a carrier 1026 which is the same as that on which sensor elements (light emitting element 1221 as a light emitting unit and a light receiving element 1222 as a light receiving unit) are disposed. In other words, the additional electronic apparatuses are disposed on a carrier different from the carrier on which the sensor elements are disposed, or a substrate. With such a configuration, it is possible to mount necessary additional electronic apparatuses on the heart rate monitoring apparatus 1050 while maintaining a satisfactory contact between the skin and the sensor elements (light emitting element 1221 and the light receiving element 1222). For example, the external electric connection terminal can be disposed on the side surface of the carrier 1026.

As described above, different types of sensors can be used in the biological information measuring apparatus according to the invention. For example, when the light receiving element 1222 mentioned above is an electric sensor, two skin conductance electrodes (for example, sensor elements (the light emitting element 1221 and the light receiving element 1222 which are illustrated in FIG. 19)) which come into contact with the skin of a user and measure the conductivity of the user are covered with the skin. Meanwhile, two or more types of sensors can be used in such a type of biological information measuring apparatus, and the number of sensor elements does not matter.

In the second to fifth embodiments, a flow chart of a method of manufacturing the proposed biological information measuring apparatus that measures a physiologic parameter is illustrated in FIG. 23.

In first step S1, the sensor 1022 including at least two sensor elements (the light emitting element 1221 and the light receiving element 1222) for detecting a sensor signal is disposed on the carrier 1026. In second step S2, an electrical contact between the sensor elements is formed in the carrier 1026. In third step S3, one or more frames 1041 and 1042 is formed on the carrier 1026 in the vicinity of the sensor 1022 and/or the individual sensor elements (the light emitting element 1221 and the light receiving element 1222). In fourth step S4, the insulating material 1032 is injected into and filled in regions surrounded by the respective frames 1041 and 1042 so as not to cover the upper surfaces 1221a and 1222a of the sensor elements (the light emitting element 1221 and the light receiving element 1222) which are provided on the carrier 1026.

According to the second to fifth embodiments described above, a method of protecting an electrical contact that does not exert a bad influence on the performance of the biological information measuring apparatus is proposed. The biological information measuring apparatus is formed by such a method as that in which the performance of a sensor is maintained. For example, at least one of the frames 1041 and 1043 prevents the position of the sensor with respect to the skin from being shifted. Further, at least one of the frames 1041 and 1043 can help emitted direct light to be prevented from being incident on the light receiving element 1222. It is preferable that the heights of the frames 1041 and 1043, facing the light receiving element 1222, in the vicinity of the respective light emitting elements 1221 and 1223 have to be smaller than the heights of the upper surfaces 1221a and 1223a of the respective light emitting elements 1221 and 1223. In addition, the frame 1042 in the vicinity of the light receiving element 1222 may be higher than the upper surface 1222a of the light receiving element 1222.

Also in the biological information measuring apparatuses according to the second to fifth embodiments described above, it is possible to apply a configuration of an interval between the light emitting unit and the light receiving unit described in the first embodiment. With such a configuration, it is possible to obtain the same effects as those in the first embodiment.

### Sixth Embodiment

The biological information measuring apparatuses of the first to fifth embodiments described above may include various types of sensors such as, a strain gauge, a thermometer, a clinical thermometer, an acceleration sensor, a gyro sensor, a piezoelectric sensor, a pressure sensor, a sphygmomanometer, an electrochemical sensor, a global positioning system (GPS), and a vibrometer. The biological information measuring apparatuses include these sensors, and thus it is possible to derive information regarding a personal physiological state on the basis of data indicating one or one or more physiological parameters, such as heartbeat, pulse, a variation between pulsations, an electrocardiogram (EKG), an electrocardiogram (ECG), a respiration rate, a skin temperature, a body temperature, a body heat flow, a galvanic skin response, a galvanic skin reflex (GSR), an electromyogram (EMG), an electroencephalogram (EEG), an electrooculography (EOG), blood pressure, body fat, a hydration level, an activity level, a body motion, oxygen consumption, glucose, a blood glucose level, muscle mass, pressure applied to a muscle, pressure applied to a bone, ultraviolet absorption, a sleep state, a physical condition, a stress state, and a posture (for example, lying, standing upright, and sitting). In addition, values obtained by the various types of sensors are transmitted to, for example, a portable communication terminal such as a smartphone, a mobile phone, or a feature phone, or an information processing terminal such as a computer or a tablet computer, so that the portable communication terminal or the information processing terminal may execute the arithmetic processing of the physiological parameters.

A user inputs his or her own profile to the biological information measuring apparatus, the portable communication terminal, or the information processing terminal before measuring biological information. Thereby, the user can receive user's unique characteristic information and environmental information which are required to be coped with, in order to maximize a possibility of a recommended healthy lifestyle being established and maintained, on the basis of the profile and biological information measurement results. Examples of information to be provided include one or two or more of exercise information such as an exercise type, an exercise strength, and an exercise time, meal information such as a meal time, the amount of meal, recommended intake ingredients and intake menus, and intake ingredients and intake menus that should be avoided, life support information such as a sleep time, the depth of sleep, the quality of sleep, a wake-up time, a landing time, a working time, stress information, consumed calories, intake calories, and calorie balance, physical information such as basal metabolism, the amount of body fat, a body fat percentage, and muscle mass, medication information, supplement intake information, and medical information.

Examples of the user's own profile include one or two or more of the age, the date of birth, the sex, hobbies, an occupation type, a blood type, a past sports history, an activity level, meal, the regularity of sleep, the regularity of bowel habit, situation adaptability, durability, responsiveness, the strength of reaction, user's personality such as a temper, a user's self-independence level, independent formation, self-management, sociability, a memory and an academic attainment ability, a user's awakening level, a perception speed, an ability to avoid attention alienation factors, user's attention including an awakening state and a self-supervision ability, an attention continuance ability, the weight, the height, blood pressure, a user's health state, medical examination results by a doctor, the date of a medical examination by a doctor, the presence or absence of a contact between a doctor and a health care person, medicines and supplements that are currently taken, the presence or absence of an allergy, an allergy history, the current allergy symptoms, an opinion of behavior pertaining to health, a user's disease history, a user's operation history, a family medical history, a social phenomenon, such as a divorce or unemployment, which is required to be adjusted by an individual, conviction pertaining to a user's health priority, a sense of values, an ability to change behavior, a phenomenon considered to be a cause of the stress of life, a stress management method, the degree of user's own consciousness, the degree of user's empathy, the degree of user's authority transfer, user's pride, user's exercise, a sleep state, a relaxed state, the current routine of daily activity, the personality of an important person in user's life (for example, a spouse, a friend, a colleague, or a superior officer), and a user's way to catch whether a conflict that disturbs a healthy lifestyle or contributes to stress is present in a relationship with an important person.

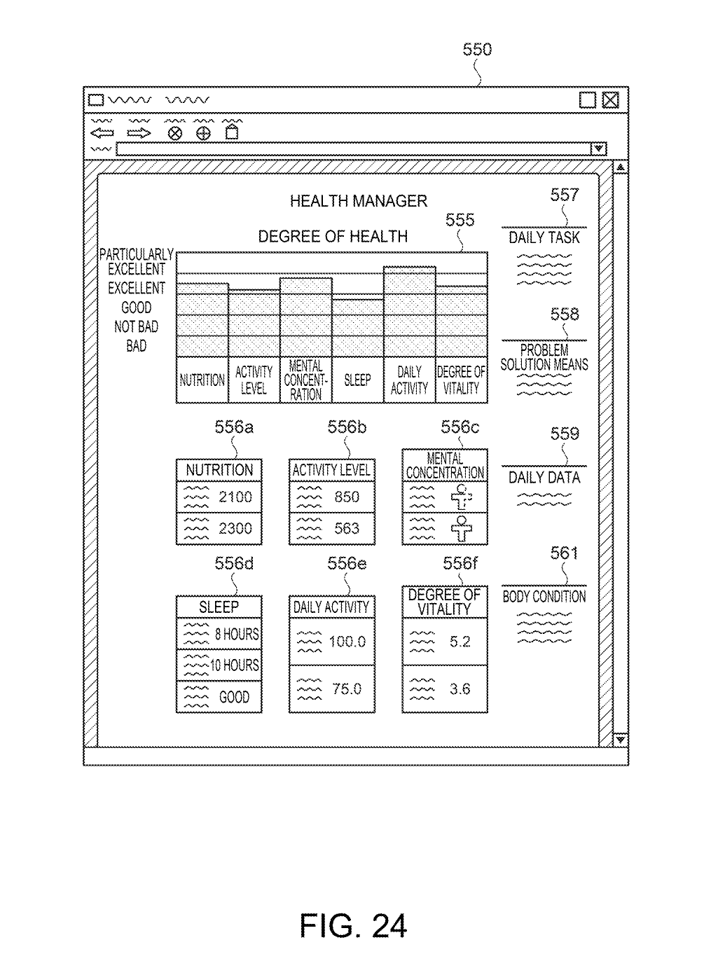

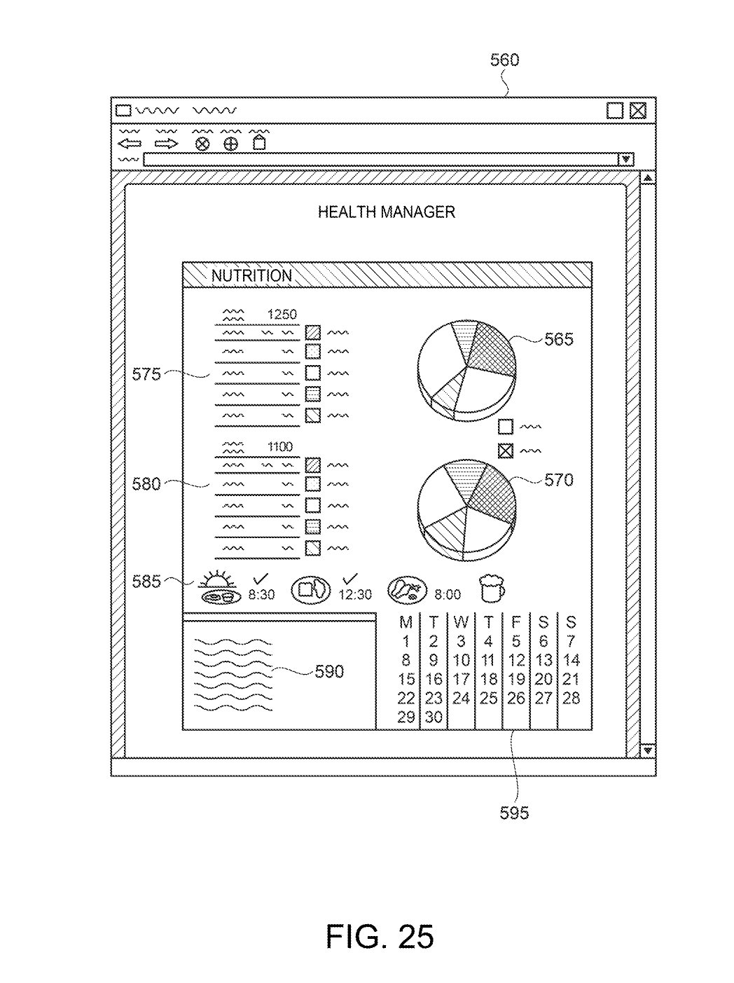

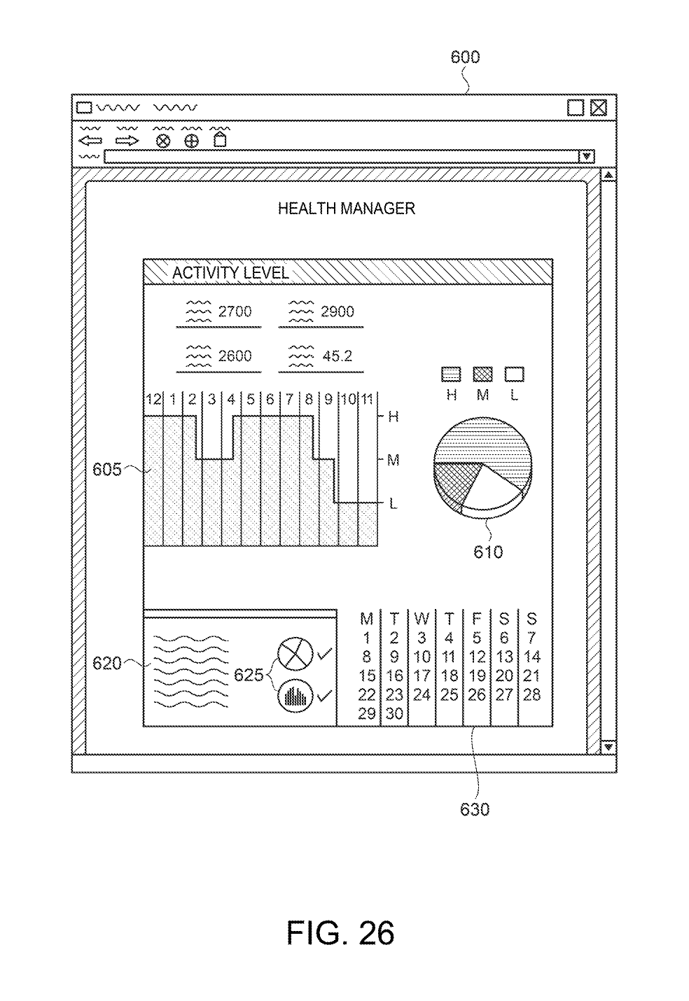

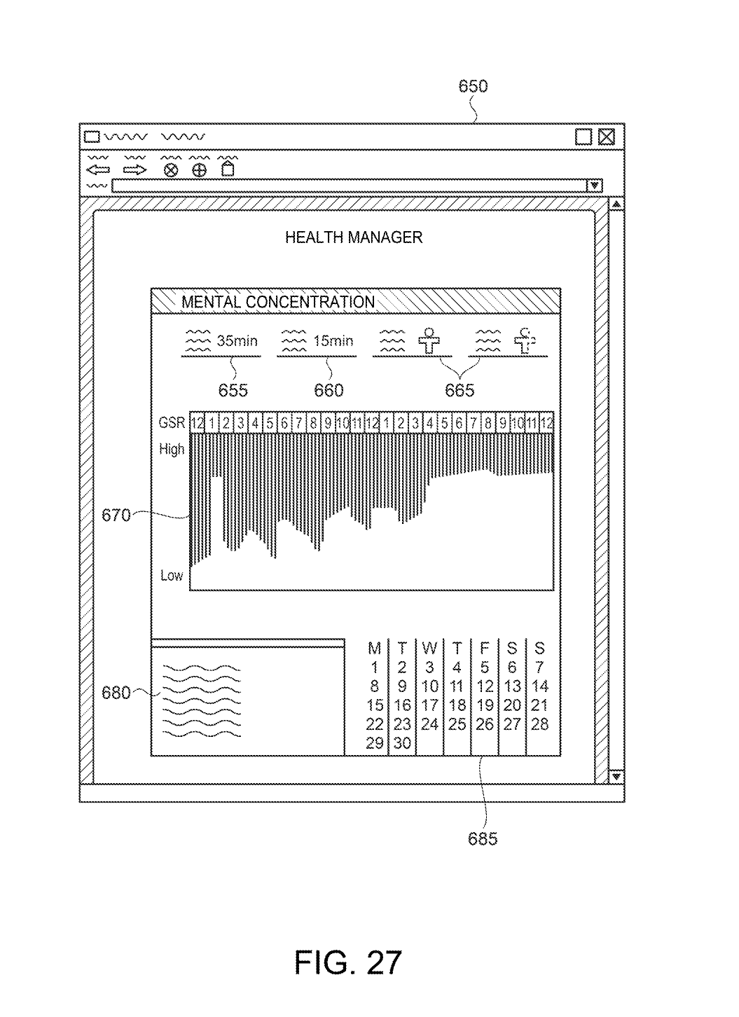

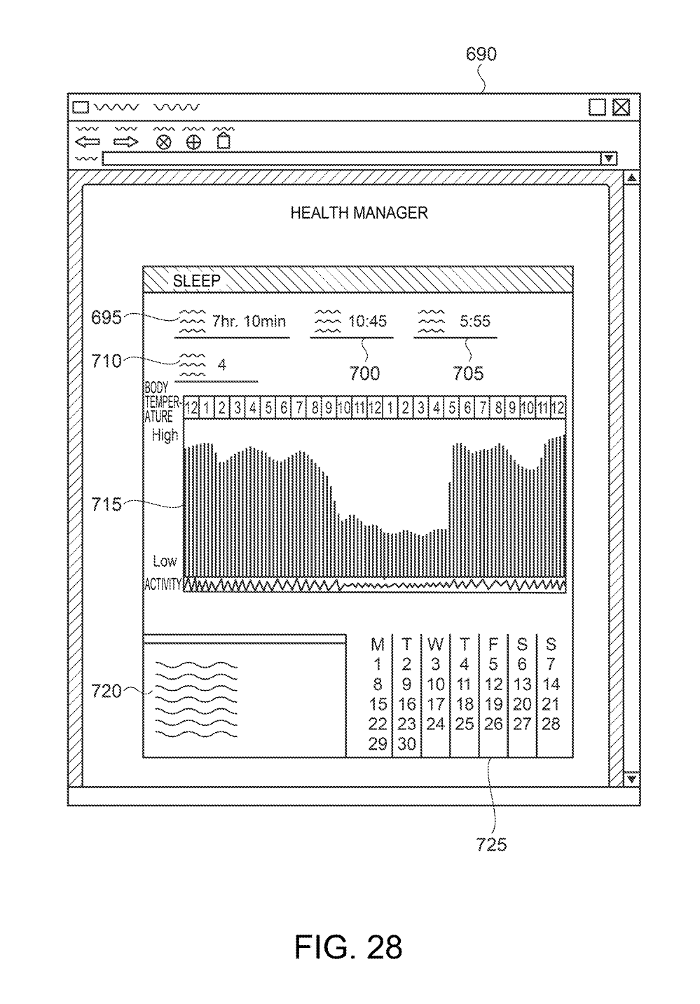

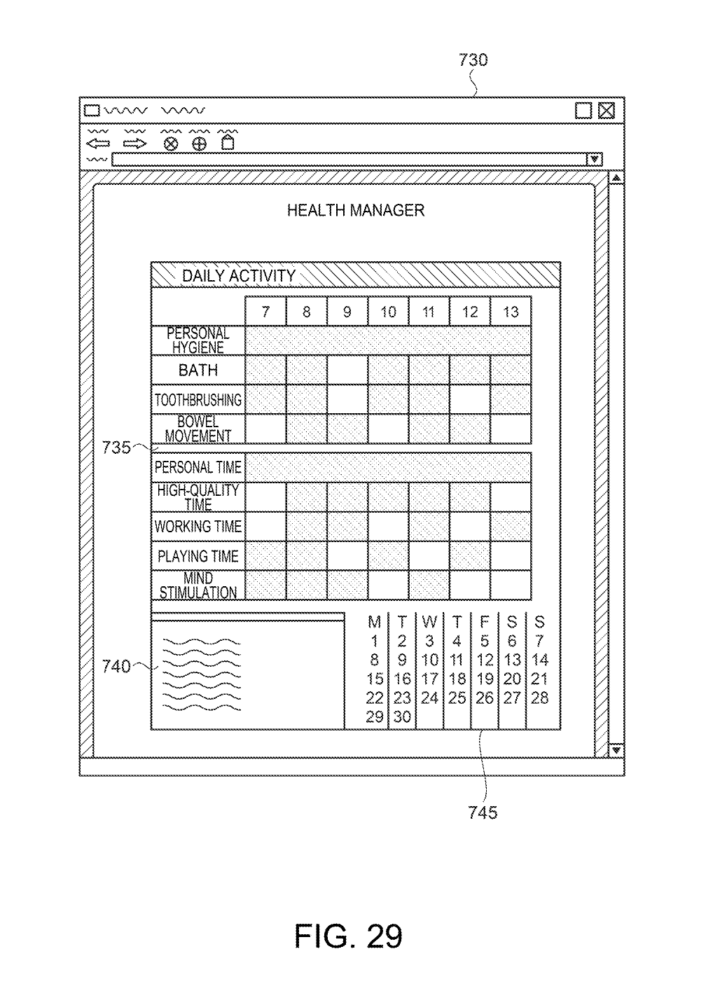

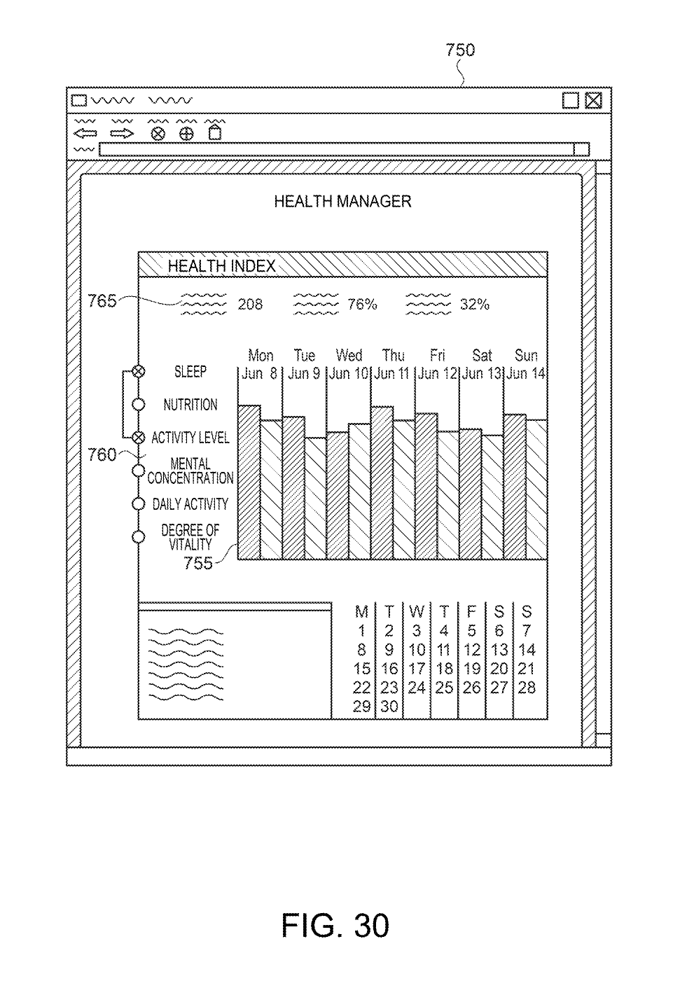

Here, reference will be made to FIGS. 24 to 30 to describe a biological information measuring apparatus according to a sixth embodiment which is capable of receiving user's unique characteristic information and environmental information which are required to be coped with, in order to maximize a possibility of a recommended healthy lifestyle being established and maintained. FIG. 24 is a schematic diagram illustrating a web page serving as a starting point of a health manager in the biological information measuring apparatus of the sixth embodiment. FIG. 25 is a diagram illustrating an example of a nutrition web page, and FIG. 26 is a diagram illustrating an example of an activity level web page. In addition, FIG. 27 is a diagram illustrating an example of a mental concentration web page, and FIG. 28 is a diagram illustrating an example of a sleep web page. In addition, FIG. 29 is a diagram illustrating an example of a daily activity web page, and FIG. 30 is a diagram illustrating an example of a health degree web page.

Although not shown in the drawing, the biological information measuring apparatus according to the sixth embodiment includes, for example, a sensor device which is connected to a microprocessor. In the biological information measuring apparatus according to the sixth embodiment, pieces of data regarding various life activity items which are finally transmitted to a monitor unit and stored, and personal data or living information which is input by a user from a website maintained by the monitor unit are processed by the microprocessor and are provided as biological information. Hereinafter, a specific example will be described.

A user has access to a health manager for the user through a web page, application software, and other communication media. FIG. 24 illustrates a web page 550 serving as a starting point of the health manager, as an example. In the web page 550 of the health manager shown in FIG. 24, various pieces of data are provided to a user. The provided data is one or more pieces of data of, for example, (1) data indicating various physiological parameters based on values measured by various sensor devices, (2) data derived from data indicating various physiological parameters, and (3) data indicating various context parameters generated by the sensor device and data input by the user.

Analysis state data has features that a certain utility or algorithm is used in order to perform conversion into (1) data indicating various physiological parameters acquired by the sensor device, (2) data derived from various physiological parameters, (3) the degree of health obtained by calculating one or more pieces of data of data indicating various context parameters acquired by the sensor device and data input by the user, (4) the degree of good health and a lifestyle index, and the like. For example, it is possible to calculate the amounts of calories, protein, fat, carbohydrates, and certain vitamin on the basis of data input by the user in relation to food taken. In addition, as another example, it is possible to provide indexes of stress levels over a desired period of time to the user by using a skin temperature, a heart rate, a respiration rate, a heat flow and/or a GSR. As still another example, it is possible to provide indexes of sleep patterns over a desired period of time to the user by using a skin temperature, a heat flow, a variation between pulsations, a heart rate, pulse, a respiration rate, a central body temperature, a galvanic skin response, an EMG, an EEG, an EOG, blood pressure, oxygen consumption, ambient sounds, and body motion detected by a device such as an accelerometer.

In the web page 550 illustrated in FIG. 24, a health index 555 as the degree of health is displayed. The health index 555 is a graphic utility for measuring the degree of achievement of user's results and a recommended healthy daily task and giving feedback to member users. In this manner, the health index 555 indicates health states and progress conditions of action pertaining to health maintenance of the member users. The health index 555 includes six categories regarding the health and lifestyle of a user, that is, nutrition, an activity level, mental concentration, sleep, daily activity, and the degree of vitality (overall impression). The category of “nutrition” pertains to information regarding what, when, and how much the person (user) has eaten and taken. The category of “activity level” pertains to the amount of exercise regarding how much the person has moved around. The category of “mental concentration” pertains to the quality (ability) of the activity for making the person (user) set to be in a relaxed state in a state where the mind of the person is in a highly concentrated state, and to a period of time for which the person concentrates on the activity. The category of “sleep” pertains to the quality and amount of sleep of the person (user). The category of “daily activity” pertains to matters that have to be performed every day by the person (user) and to health risks that the person meets with. The category of “the degree of vitality (impression)” pertains to a general way to catch whether being in a good mood on a certain day. Preferably, each of the categories includes a level display or a bar graph indicating how many results the user has attained on a scale varying between “bad” and “good”.

When each member user terminates the above-mentioned initial examination, a profile for providing a user's own characteristics and a summary of a living environment to the user is created, and recommended healthy daily tasks and/or targets are presented. The recommended healthy daily tasks include any combination in specific pieces of advices regarding appropriate nutrition, exercise, mental concentration, and user's daily activity (life). A model schedule or the like may be presented as a guide indicating how to take activity items pertaining to the recommended healthy daily tasks in the user's life. The user is regularly subjected to the examination, and practices the above-mentioned items accordingly on the basis of the results thereof.

The category of “nutrition” is calculated from both data input by a user and data sensed by a sensor device. The data input by the user includes the times for breakfast, lunch, and dinner, and any snack and the eating and drinking times thereof, and food to be eaten and drunk, supplements such as vitamin, and water or another liquid (drinking water or liquid food) which is drunk during a time which is selected in advance. A central monitoring unit calculates consumed calories or well-known nutritional values such as the contents of protein, fat, carbohydrates, vitamin, and the like, on the basis of the data and stored data regarding known characteristics of various articles of food.

In the category of “nutrition”, a recommended healthy daily task can be determined on the basis of the bar graph indicating the nutrition of the health index 555. The recommended healthy daily task can be adjusted on the basis of information such as the sex, age, and height/weight of a user. Meanwhile, a user or a representative of the user can set a target of certain nutrition pertaining to the amount of calories consumed every day, the amount of nutriments such as protein, fiber, fat, and carbohydrates, the amount of water, and ratios thereof to the total intake. Parameters used for the calculation of the bar graph include the number of meals for one day, the amount of water consumed, and the type and amount of food eaten every day which are input by a user.

Nutritional information is presented to a user by a nutrition web page 560 as illustrated in FIG. 25. It is preferable that the nutrition web page 560 includes nutrition numerical charts 565 and 570 that are pie charts showing actual and target numerical values of nutrition, and nutrition intake charts 575 and 580 showing an actual total nutrition intake amount and a target total nutrition intake amount. In the nutrition numerical charts 565 and 570, it is preferable that items such as carbohydrates, protein, and fat are expressed by percentage. In the nutrition intake charts 575 and 580, it is preferable that a total value and a target value of calories are expressed by being divided into ingredients such as fat, carbohydrates, protein, and vitamin. The nutrition web page 560 includes a history 585 indicating the times when food and water are consumed, a hyperlink 590 that allows a user to be able to directly check a news story pertaining to nutrition, advice for improving a daily task pertaining to nutrition, and any related advertisement on a network, and a calendar 595 in which an application period and the like can be selected. Items indicated by the hyperlink 590 can be selected on the basis of information learned from an individual through examination, and the individual's results measured by the health index.

The category of “activity level” in the health index 555 is designed so as to support a user's check regarding when and how the user acted (moved) on that day, and the like, and both data input by the user and data sensed by the sensor device are used. The data input by the user includes details pertaining to the user's daily activity such as, for example, doing work at the desk from 8 a.m. to 5 p.m. and taking an aerobic lesson from 6 p.m. to 7 p.m. The related data sensed by the sensor device includes a heart rate, an exercise sensed by a device such as an accelerometer, a heat flow, a respiration rate, the amount of calories consumed, a GSR, and a water supply level, and these can be taken out by the sensor device or the central monitoring unit. The amount of calories consumed can be calculated by various methods such as multiplication of the type of exercise which is input by the user and the duration of exercise which is input by the user, multiplication of the sensed exercise, an exercise time, and a filter constant, or multiplication of the sensed heat flow, the time, and a filter constant.

In the category of “activity level”, a recommended healthy daily task can be determined on the basis of the bar graph indicating the activity level of the health index 555. The recommended healthy daily task includes a minimum target calories consumed by the activity, and the like. Meanwhile, the minimum target calories can be set on the basis of information such as the sex, age, height, and weight of a user. Parameters used for the calculation of the bar graph includes a time input by the user and/or a time sensed by the sensor device which are times spent for various types of exercises or an energetic lifestyle activity, and the amount of calories burned over an energy consumption parameter which is calculated in advance.

Information regarding the activity (movement) of an individual user is presented to the user by an activity level web page 600 illustrated in FIG. 26. The activity level web page 600 includes an activity degree graph 605, having a bar graph shape, which shows the user's activity monitored according to three categories, that is, “high”, “medium”, and “low” that are classified with respect to a predetermined unit time. An activity percentage chart 610 having a pie chart shape can be presented in order to express a percentage for a predetermined period of time such as, for example, one day which is spent in each of the categories by the user. In addition, the activity level web page 600 may include a calorie display (not shown) for displaying items such as a total amount of calories burned, a target value of daily burned calories, a total value of calories taken, and an aerobic exercise time. The activity level web page 600 includes at least one hyperlink 620 in order to allow the user to be able to directly check a related news story, advice for improving a daily task pertaining to an activity level, and a related advertisement on a network.

The activity level web page 600 can be viewed in various formats, and can be configured such that a user can select a bar graph, a pie chart, or both the graph and the chart and the selection can be performed by an activity level check box 625. An activity level calendar 630 is provided so that an application period and the like can be selected. Items indicated by the hyperlink 620 can be selected on the basis of information extracted from an individual through examination, and the results measured by the health index.

The category of “mental concentration” in the health index 555 is designed so as to support a user's monitoring of parameters pertaining to a time when the activity for allowing the user's body to reach a deep relaxed state while concentrating his or her mind is performed, and is based on both data input by the user and data sensed by the sensor device. In detail, the user can input a starting time and a termination time of a relaxation activity such as yoga or meditation. The quality of these activity items determined by the depth of mental concentration can be measured by monitoring parameters including a skin temperature, a heart rate, a respiration rate, and a heat flow which are sensed by the sensor device. It is also possible to use a variation in the percentage of a GSR obtained by either of the sensor device or the central monitoring unit.

In the category of “mental concentration”, a recommended healthy daily task can be determined on the basis of the bar graph indicating the activity level of the mental concentration in the health index 555. The recommended healthy daily task is displayed inclusive of daily joining in the activity of deeply relaxing a body while making mind set to be in a highly concentrated state. Parameters used for the calculation of the bar graph include the length of time spent for the mental concentration activity, the depth of the mental concentration activity, or a variation in the percentage of a skin temperature, a heart rate, a respiration rate, a heat flow, or a GSR which is sensed by the sensor device from a base line indicating quality.

Information regarding time spent for an action of deeply looking back oneself (introspection) and for mental concentration activity such as deep relaxation of a body is presented to a user by a mental concentration web page 650 illustrated in FIG. 27. Meanwhile, the mental concentration activity may be referred to as a session. The mental concentration web page 650 includes a time 655 spent for the session, a target time 660, comparison portions 665 indicating a target value of the depth of mental concentration and an actual value, and a histogram 670 indicating the overall stress level which is derived from a skin temperature, a heart rate, a respiration rate, a heat flow, and/or a GSR.

In the comparison portion 665, the contour of a human indicating a target mental concentration state is shown by a solid line, and the contour of a human indicating an actual mental concentration state varies between a blurred state (shown by a dashed line in FIG. 27) and a solid line in accordance with the level of mental concentration. In addition, the preferable mental concentration web page 650 includes a hyperlink 680 that allows a user to be able to directly check a related news story, advice for improving a daily task pertaining to mental concentration, and a related advertisement on a network, and a calendar 685 in which advice for improving a daily task pertaining to mental concentration, a related advertisement and an application period can be selected. Items indicated by the hyperlink 680 can be selected on the basis of results measured by information learned from an individual through examination, and the results measured by the health index.

The category of “sleep” in the health index 555 is designed so as to be able to support a user's monitoring of a sleep pattern and the quality of sleep. This category is intended to help a user to learn the importance of sleep in a healthy lifestyle and the relation of sleep to a daily cycle which is an ordinary daily variation in the function of the body. The category of “sleep” is based on both data input by the user and data sensed by the sensor device. The data input by the user between related time intervals includes ranks of a sleep-onset time and a wake-up time (sleep time) of the user and the quality of sleep. The related data obtained by the sensor device includes a skin temperature (body temperature), a heat flow, a variation between pulsations, a heart rate, a pulse rate, a respiration rate, a central body temperature, a galvanic skin response, an EMG, an EEG, an EOG, blood pressure, and oxygen consumption. In addition, ambient sounds and body motion which is detected by a device such as an accelerometer also have relevance. Thereafter, a sleep-onset time, a wake-up time, the interruption of sleep, the quality of sleep, the depth of sleep, and the like can be calculated and derived using the data.

The bar graph showing the sleep in the health index 555 displays a healthy daily task including the securing of a preferable nightly minimum sleep time, a predictable bedtime, and a wake-up time. Specific parameters enabling the calculation of the bar graph include a daily sleep time and a wake-up time which are sensed by the sensor device or input by the user, and the quality of sleep which is graded by the user or derived from another data.

Information regarding the sleep is presented to a user by a sleep web page 690 illustrated in FIG. 28. The sleep web page 690 includes a sleep time display 695 based on either of data from the sensor device or data input by the user, a user bedtime display 700, and a wake-up time display 705. Meanwhile, the quality of sleep which is input by the user can be displayed using a sleep quality rank 710. In addition, when a display exceeding a time interval for one day is performed in the sleep web page 690, the sleep time display 695 can be displayed as a cumulative value, and the bedtime display 700, the wake-up time display 705, and the sleep quality rank 710 can be calculated and displayed as average values. In addition, the sleep web page 690 also includes a sleep graph 715 selectable by a user who calculates and displays one sleep-related parameter during a predetermined time interval. FIG. 28 illustrates a variation in a heat flow (body temperature) for one day. The heat flow tends to be reduced while asleep and to be increased while awake. It is possible to obtain a biorhythm of the person from the information.

In addition, the sleep graph 715 graphs data from an accelerometer embedded in the sensor device that monitors body motion. In addition, the sleep web page 690 can include a hyperlink 720 that allows a user to be able to directly check a news story pertaining to sleep, advice for improving a daily task pertaining to sleep, and a related advertisement on a network, and a sleep calendar 725 for selecting a related time interval. Items indicated by the hyperlink 720 can be particularly selected on the basis of information learned from an individual in examination, and results measured by the health index.

The category of “daily activity” in the health index 555 is designed so as to be able to support a user's monitoring of a certain activity, pertaining to health or safety, and risk, and is completely based on data input by a user. The category of “daily activity” pertaining to activity in a daily life includes four categories which are subordinate concepts. Specifically, the category is classified into (1) an item pertaining to personal hygiene which enables a user's monitoring of dental care using a toothbrush or floss or activity such as taking a shower, (2) an item pertaining to health maintenance which enables tracing of whether a user is taking medicine or a supplement as prescribed, and enables a user's monitoring of the consumption of cigarettes or alcohol, and the like, (3) an item pertaining to personal time which enables a user's monitoring of time or leisure, which is spent with the user's family or friend, and mental concentration activity, and (4) an item pertaining to responsibility which enables a user's monitoring of work, such as household chores, and household activity.

In the category of “daily activity”, it is preferable that the bar graph indicating the “daily activity” in the health index 555 displays the following recommended healthy daily tasks. As an example of a daily task pertaining to the personal hygiene, it is preferable that a user takes a shower or takes a bath every day, keeps his or her teeth clean by using a toothbrush or floss every day, and has regular bowel movements. In addition, as an example of a daily task pertaining to the health maintenance, it is preferable that a user takes medicine, vitamin pills, and/or supplements, does not smoke, drinks in moderation, and monitors his or her health every day by a health manager. As an example of a daily task pertaining to the personal time, it is preferable that a user makes at least predetermined time every day in order to spend the time with his or her family, and/or spends high-quality time with his or her friend, reduces time for work, takes time for leisure or play, and performs activity using his or her brain. As an example of a daily task pertaining to the responsibility, it is preferable that a user does household chores, is not late for work, and keeps a promise. The bar graph is determined by information input by a user, and/or is calculated on the basis of the degree to which the user completes activity listed up every day.

Pieces of information regarding these activity items are presented to a user by a daily activity web page 730 illustrated in FIG. 29. An activity chart 735 in the daily activity web page 730 shows whether a user has executed necessary activity by the daily task. In the activity chart 735, one or more of the subordinate concepts can be selected. In the activity chart 735, a box which is colored or shaded indicates that a user has executed necessary activity, and a box which is not colored or shaded indicates that the user has not executed the activity. The activity chart 735 can be created at a selectable time interval and can be viewed. FIG. 29 illustrates the categories of personal hygiene and personal time in a specific week as an example. Further, the daily activity web page 730 may include a hyperlink 740 that allows a user to be able to directly check a related news story, advice for improving a daily task pertaining to activity in a daily life, and a related advertisement on a network, and a daily activity calendar 745 for selecting a related time interval. Items indicated by the hyperlink 740 can be selected on the basis of information learned from an individual in examination, and results determined by the health index.

The category “the degree of vitality” in the health index 555 is designed so as to enable a user's monitoring of recognition of whether being in good spirits on a specific day, and is based on essentially subjective grade information which is directly input by the user. The user performs ranking using scales of, preferably, 1 to 5 with respect to the following nine areas, that is, (1) mental keenness, (2) the degree of mental and psychological happiness, (3) an energy level, (4) a capacity for stresses of life, (5) the degree of being concerned about appearances, (6) the degree of physical happiness, (7) self-control, (8) a motive, and (9) comfort by a relationship with others. These degrees (grades) are averaged to be used for the calculation of the bar graph of the health index 555.

FIG. 30 illustrates a vitality degree web page 750. The vitality degree web page 750 allows a user to be able to check the degree of vitality during a time interval, selectable by the user, which includes continuous or discontinuous arbitrary days. Meanwhile, in the example illustrated in FIG. 30, the degree of vitality is displayed as a health index. In the vitality degree web page 750, a user can perform selection for checking a vitality degree bar graph 755 with respect to one category or can compare the vitality degree bar graphs 755 in parallel with respect to two or more categories by using the vitality degree selection box 760. For example, the user may set only a bar graph for sleep to be in an operation state in order to check whether the overall grade of sleep has been improved compared to the previous month, or may compare the grade of sleep with the grade of an activity level corresponding thereto and evaluates the grades by simultaneously displaying the sleep and the activity level and may check whether there is some correlation between the days. The grade of nutrition and the grade of the degree of vitality may be displayed for a predetermined time interval so that it is checked whether there is some correlation between a daily dietary habit, a dietary habit during the interval, and the degree of vitality. FIG. 30 illustrates comparison between sleep and an activity level during a week from June 8 to June 14 using bar graphs, as an example for description. In addition, the vitality degree web page 750 also includes a tracing calculator 765 that displays access information, such as the sum of days in which a user has logged on and used the health manager, the proportion of days in which the user has used the health manager since admission, and the proportion of hours for which the user has used the sensor device in order to collect data, and statistics.

An example of the web page 550 serving as a starting point of the health manager illustrated in FIG. 24 includes summaries 556a to 556f of a plurality of categories, selectable by a user, which correspond to the categories of the health index 555 as the degree of health. Each of the summaries 556a to 556f of the respective categories presents a subset of data which is selected in advance with respect to the corresponding category and is filtered. The summary 556a of the category of nutrition indicates a daily target value and an actual value of a caloric intake. The summary 556b of the category of activity level indicates a daily target value and an actual value of the amount of calories burned. The summary 556c of the category of mental concentration indicates a target value and an actual value of the depth of mental concentration. The summary 556d of the category of sleep indicates a target sleep time, an actual sleep time, and the grade of the quality of sleep. The summary 556e of the category of daily activity displays a target point and an actual point based on a ratio of completed activity to a recommended healthy daily task (daily activity). The summary 556f of the category of the degree of vitality indicates a target grade and an actual grade of the degree of health of the day.

In addition, the web page 550 may also include a hyperlink (not shown) to a news story, comments (not shown) to a user based on a tendency such as malnutrition which is checked by the first examination, and a signal (not shown). The web page may also include a daily task portion 557 that provides information to a user every day. As comments of the daily task portion 557, for example, a water intake required every day, advice for specific means for enabling the intake of water, and the like can be displayed. In addition, the web page 550 may include a problem solution section 558 that actively evaluates a user's results in each category of the health index 555 and presents advice for improvement. For example, when a user's sleep level is “low” by a system and it is suggested that the user has insomnia, the problem solution section 558 can advise a method for improving sleep. In addition, the problem solution section 558 may include the user's question regarding an improvement in results. In addition, the web page 550 may include a daily data section 559 that starts up an input dialogue box. The user can easily input various pieces of data required by the health manager, using the input dialogue box. As known in the art, the input of data can be selectively performed between the input in a list presented in advance and the input in a general free text format. In addition, the web page 550 may include a body condition section 561 that gives information regarding life symptoms such as the height and weight of a user, a body measurement value, a BMI, a heart rate, blood pressure, or any physiological parameter.

### Modification Example of Light Receiving Unit

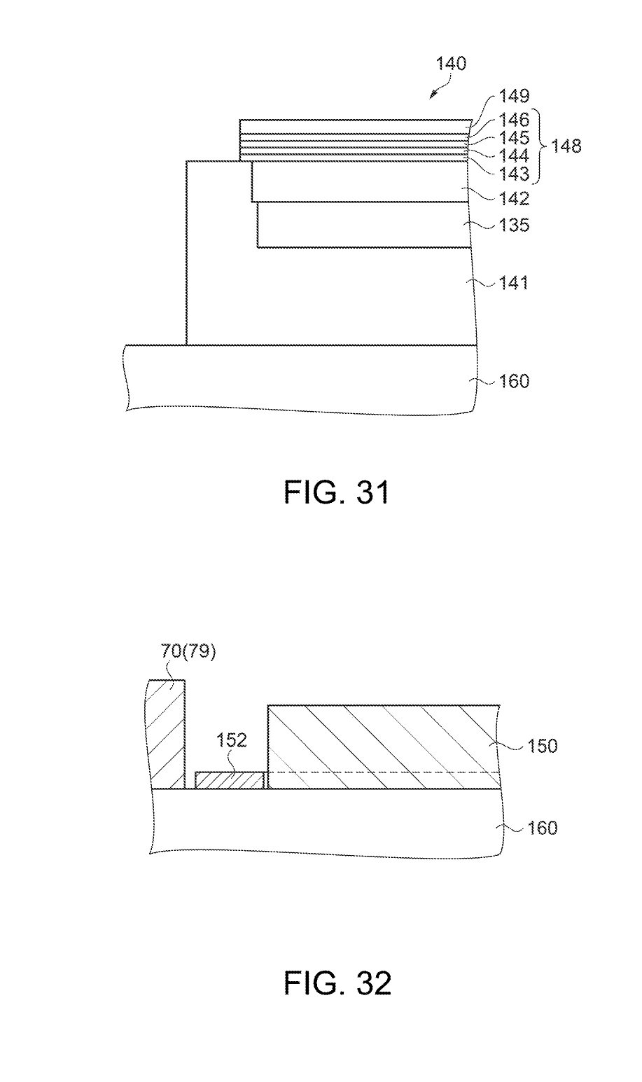

Here, a modification example of the light receiving unit 140 according to the first embodiment described above will be described with reference to FIG. 31. FIG. 31 is a partial cross-sectional view illustrating a modification example of a light receiving unit. As illustrated in FIG. 31, the light receiving unit 140 mounted on a substrate 160 (sensor substrate) can be realized by a photodiode element 135 of a PN junction which is formed on a semiconductor substrate 141, and the like. In this case, an angle limiting filter for narrowing a light reception angle or a multilayered film optical filter 148 functioning as a wavelength limiting filter that limits a wavelength of light incident on a light receiving element may be formed on the upper side of the photodiode element 135, the angle limiting filter 142, and the like. Meanwhile, the multilayered film optical filter 148 is configured such that, for example, a first oxide film 143, a first nitride film 144, a second oxide film 145, and a second nitride film 146 are formed in this order from the angle limiting filter 142 side. A resin film 149 having light transmittance is provided on the multilayered film optical filter 148.

With such a configuration, it is possible to increase a waterproofing property and an antifouling property of the multilayered film optical filter (wavelength limiting filter) 148 by the resin film 149, having light transmittance, which is provided on the multilayered film optical filter 148.

Meanwhile, this modification example of the light receiving unit can be applied to any of the above-described embodiments or configuration examples.

### Modification Example of Light Emitting Unit

Next, a modification example of the light emitting unit 150 according to the first embodiment described above will be described with reference to FIG. 32. FIG. 32 is a partial cross-sectional view illustrating a modification example of a light emitting unit. As illustrated in FIG. 32, a wall portion 70 as a frame, and a reflective functional layer 152 that reflects light emitted in a peripheral direction from a light emitting unit 150 are provided in the vicinity of the light emitting unit 150 mounted on a substrate 160 (sensor substrate). Meanwhile, the reflective functional layer 152 may be provided so as to surround the vicinity of the light emitting unit 150 over the whole periphery or may be provided in at least a portion of the vicinity of the light emitting unit 150 in a plan view when seen from the upper surface side of the substrate 160.

With such a configuration, light emitted in a peripheral direction of the light emitting unit 150 can be made to be reflected by a reflective functional layer 152 and to be directed to a measurement object. Thereby, it is possible to increase the intensity (light emission intensity) of light directed to the measurement object, and to improve and stabilize the measurement accuracy of biological information.

Meanwhile, this modification example of the light receiving unit can be applied to any of the above-described embodiments or configuration examples.

Meanwhile, embodiments of the invention have been described above in detail, but those skilled in the art may easily understand that many variations are conceivable to the extent that they do not substantially depart from the novel items and effects of the invention. Therefore, such variations all fall within the scope of the invention. For example, a term described at least once in the specification or the drawings with a different term having a broader meaning or the same meaning can be replaced with the different term anywhere in the specification or the drawings. Further, the configuration and action of each of the biological information measuring module, the light detection unit, the biological information measuring apparatus, and the like are not limited to those described in this embodiment of the invention, and a variety of changes can be made thereto.

What is claimed is:

1. A biological information measuring module comprising:
a light emitting unit that emits light;
a light receiving unit that receives the light via an object; and
a multilayered film optical filter that is provided on the light receiving unit on a side where the light is incident,
wherein the multilayered film optical filter is constituted by a laminated body of five or more layers and 120 or less layers.

2. The biological information measuring module according to claim 1, wherein the multilayered film optical filter is constituted by a laminated body of ten or more layers and 120 or less layers.

3. The biological information measuring module according to claim 1, wherein the multilayered film optical filter is constituted by a laminated body of twenty or more layers and sixty or less layers.

4. The biological information measuring module according to claim 1, wherein the multilayered film optical filter is constituted by a laminated body of twenty three or more layers and sixty or less layers.

5. The biological information measuring module according to claim 1, wherein the multilayered film optical filter is constituted by a laminated body of forty or more layers and sixty or less layers.

6. The biological information measuring module according to claim 1, wherein the multilayered film optical filter is constituted by a laminated body of twenty three or more layers and forty or less layers.

7. The biological information measuring module according to claim 1, wherein a reflective functional layer is provided in at least a portion of a vicinity of the light emitting unit.

8. The biological information measuring module according to claim 1, wherein a frame is provided between the light emitting unit and the light receiving unit.

9. The biological information measuring module according to claim 8, wherein the frame is formed of a resin or a metal.

10. The biological information measuring module according to claim 1, further comprising a supporting unit,
wherein the light emitting unit and the light receiving unit are supported by a supporting surface of the supporting unit.

11. The biological information measuring module according to claim 1, wherein a light condensing member is provided on the light emitting unit on a side where the light is emitted.

12. The biological information measuring module according to claim 1, wherein a plurality of the light emitting units are provided.

13. The biological information measuring module according to claim 1, wherein a plurality of the light receiving units are provided.

14. The biological information measuring module according to claim 1, wherein the laminated body is configured such that an oxide film and a nitride film are alternately laminated on each other.

15. The biological information measuring module according to claim 14, wherein the lowermost layer of the laminated body on the light receiving unit side is an oxide film.

16. The biological information measuring module according to claim 14, wherein the lowermost layer of the laminated body on the light receiving unit side is a nitride film.

17. The biological information measuring module according to claim 1, wherein a resin film is provided on the multilayered film optical filter.

18. A biological information measuring apparatus comprising the biological information measuring module according to claim 1.

19. A light detecting apparatus comprising:
a semiconductor substrate;
a light detecting element that is formed on a surface of the semiconductor substrate;
a wiring that is formed on the semiconductor substrate; and
a multilayered film optical filter that is formed on the light detecting element and the wiring,
wherein the multilayered film optical filter is formed in such a way that the wiring is positioned inside a region in which the multilayered film optical filter is formed, when seen in a plan view in a direction perpendicular to the semiconductor substrate.

20. The light detecting apparatus according to claim 19, further comprising a pad for external connection of the light detecting apparatus,
wherein the multilayered film optical filter is formed so as to have an opening at a position of the pad, when seen in the plan view.

21. The light detecting apparatus according to claim 20, wherein the pad is a pad for connecting an end of the light detecting element to the outside.

22. The light detecting apparatus according to claim 19, further comprising a light shielding layer that is formed on the semiconductor substrate,
wherein the multilayered film optical filter is formed in such a way that the light shielding layer is positioned inside a region in which the multilayered film optical filter is formed, when seen in the plan view.

23. The light detecting apparatus according to claim 22, wherein the multilayered film optical filter is formed in such a way that one side of the multilayered film optical filter is positioned between one side of the light detecting apparatus and one side of the light shielding layer which faces the one side of the light detecting apparatus, when seen in the plan view.

24. The light detecting apparatus according to claim 20, wherein the wiring is a wiring for electrically connecting the pad and an end of the light detecting element.

25. The light detecting apparatus according to claim 19, further comprising an insulating layer,
wherein when a direction when seen in the plan view is set to be a first direction and a direction perpendicular to the first direction is set to be a second direction, the multilayered film optical filter is formed on the second direction side of the insulating layer in an end of the insulating layer.

26. The light detecting apparatus according to claim 19, wherein the multilayered film optical filter is formed so as to cover the wiring and the light detecting element when seen in the plan view.

27. A light detecting apparatus comprising:
a semiconductor substrate;
a light detecting element that is formed on a surface of the semiconductor substrate;
a light shielding layer that is formed on the semiconductor substrate; and
a multilayered film optical filter that is formed on the light detecting element and the light shielding layer,
wherein the multilayered film optical filter is formed in such a way that one side of the multilayered film optical filter is positioned between one side of the light detecting apparatus and one side of the light shielding layer which faces the one side of the light detecting apparatus, when seen in a plan view in a direction perpendicular to the semiconductor substrate.

28. A light detecting module comprising the light detecting apparatus according to claim 19.

29. An electronic apparatus comprising the light detecting apparatus according to claim 19.
